(19) United States
(12) Patent Application Publication
(10) Pub. No.: US 2020/0172473 A1
(43) Pub. Date: Jun. 4, 2020

(54) DUAL MODULATORS OF FARNESOID X RECEPTOR AND SOLUBLE EPOXIDE HYDROLASE

(71) Applicant: JOHANN WOLFGANG GOETHE-UNIVERSITÄT FRANKFURT AM MAIN, Frankfurt am Main (DE)

(72) Inventors: Daniel Merk, Frankfurt am Main (DE); Jurema Schmidt, Rodgau (DE); Ewgenij Proschak, Frankfurt am Main (DE); Manfred Schubert-Zsilavecz, Bad Homburg (DE); Moritz Helmstaedter, Frankfurt am Main (DE)

(21) Appl. No.: 16/614,785

(22) PCT Filed: May 24, 2018
(86) PCT No.: PCT/EP2018/063699

§ 371 Date: Nov. 18, 2019

(30) Foreign Application Priority Data
May 24, 2017 (EP) PCT/EP2017/062692

(51) Int. Cl. C07C 233/63; C07D 211/62; C07C 233/87; C07C 237/36; C07C 235/52; C07C 275/42; C07C 233/73; C07C 233/76; C07C 255/60; C07C 233/78; C07D 257/04; C07C 311/46; C07C 323/42; C07C 317/32; C07D 207/06

(52) U.S. Cl. CPC C07C 233/63; C07D 207/06; C07C 233/87; C07C 237/36; C07C 235/52; C07C 275/42; C07C 233/73; C07C 233/76; C07C 255/60; C07C 233/78; C07D 257/04; C07C 311/46; C07C 323/42; C07C 317/32; C07D 211/62

(57) ABSTRACT

The present invention pertains to novel dual modulators of farnesoid X receptor (FXR) and soluble epoxide hydrolase (sEH). The modulators of the invention were designed to provide compounds which harbor a dual activity as agonists of FXR and inhibitors (antagonists) of sEH. The invention also provides methods for treating subjects suffering from diseases associated with FXR and sEH, such as metabolic disorders, in particular non-alcoholic fatty liver or nonalcoholic steatohepatitis (NASH).

### FIELD OF THE INVENTION

The present invention pertains to novel dual modulators of farnesoid X receptor (FXR) and soluble epoxide hydrolase (sEH). The modulators of the invention were designed to provide compounds which harbor a dual activity as agonists of FXR and inhibitors (antagonists) of sEH. The invention also provides methods for treating subjects suffering from diseases associated with FXR and sEH, such as metabolic disorders, in particular non-alcoholic fatty liver or nonalcoholic steatohepatitis (NASH).

### DESCRIPTION

Non-alcoholic fatty liver disease (NAFLD) resulting from over-nutrition and sedentary lifestyle increasingly affects adults especially in western civilizations and all over the world. Recent estimates assume that up to every third adult in the world carries NAFLD and that up to 15% proceed to non-alcoholic steatohepatitis (NASH). Most alarmingly, up to 11% of adolescents are thought to be affected by NAFLD. The disease complex of NAFLD and NASH—often considered as hepatic manifestation of the metabolic syndrome—therefore constitutes a serious health thread. However, current NAFLD treatment is limited and pharmacological options are insufficient Hence, novel pharmacological interventions to treat NAFLD and NASH are urgently required (Rinella, M. E. Nonalcoholic Fatty Liver Disease: A Systematic Review. JAMA 2015, 313 (22), 2263-2273; Gawrieh, S.; Chalasani, N. Pharmacotherapy for Nonalcoholic Fatty Liver Disease. Semin. Liver Dis. 2015, 35 (3), 338-348; Chalasani, N.; Younossi, Z.; Lavine, J. E.; Diehl, A. M.; Brunt, E. M.; Cusi, K.; Charlton, M.; Sanyal, A. J. The Diagnosis and Management of Non-Alcoholic Fatty Liver Disease: Practice Guideline by the American Association for the Study of Liver Diseases, American College of Gastroenterology, and the American Gastroenterological Association. Hepatology 2012, 55 (6), 2005-2023).

In vivo models and clinical trials have identified several strategies with therapeutic potential for the treatment of NAFLD/NASH. Particularly the nuclear farnesoid X receptor (FXR) (Arab, J. P.; Karpen, S. J.; Dawson, P. A.; Arrese, M.; Trauner, M. Bile Acids and Nonalcoholic Fatty Liver Disease: Molecular Insights and Therapeutic Perspectives. Hepatology 2017, 65 (1), 350-362) seems a very promising target for NAFLD/NASH therapy since obeticholic acid (6α-ethyl-CDCA, OCA, 1a) which was developed from the endogenous FXR agonist chenodeoxycholic acid (CDCA, 1b) has already revealed clinical efficacy in NASH treatment (Adorini, L et al. Farnesoid X Receptor Targeting to Treat Nonalcoholic Steatohepatitis. Drug Discov. Today 2012, 17 (17-18), 988-997; Neuschwander-Tetri, B. et al. Farnesoid X Nuclear Receptor Ligand Obeticholic Acid for Non-Cirrhotic, Non-Alcoholic Steatohepatitis (FLINT): A Multicentre, Randomised, Placebo-Controlled Trial. Lancet 2014, 385 (9972), 956-965). FXR is a ligand-activated transcription factor mainly found in liver, intestine and kidney and is physiologically activated by bile acids (Maldshima, et al. Identification of a Nuclear Receptor for Bile Acids. Science 1999, 284 (5418), 1362-1365; Parks, D. J. et al. Bile Acids: Natural Ligands for an Orphan Nuclear Receptor. Science 1999, 284 (5418), 1365-1368; Wang, H. et al. Endogenous Bile Acids Are Ligands for the Nuclear Receptor FXR/BAR. Mol. Cell 1999, 3 (5), 543-553). It acts as metabolic regulator by controlling several genes involved in bile acid, lipid and glucose homeostasis and as an important liver protector by sensing toxic levels of accumulating bile acids (Gadaleta, R. M et al. Tissue-Specific Actions of FXR in Metabolism and Cancer. Biochim. Biophys. Acta 2014, 1851, 30-39; Pellicciari, R. et al; Farnesoid X Receptor: From Structure to Potential Clinical Applications. J Med Chem 2005, 48 (17), 5383-5403).

Clinical trials have reported improved histological features and clinical markers of NAFLD/NASH as well as improved metabolic parameters after OCA (1a) treatment which has validated FXR as target to treat fatty liver disorders and, potentially, other metabolic diseases (Mudaliar, S et al. Efficacy and Safety of the Farnesoid X Receptor Agonist Obeticholic Acid in Patients with Type 2 Diabetes and Nonalcoholic Fatty Liver Disease. Gastroenterology 2013, 145 (3), 574-582.e1). However, full activation of FXR might cause undesirable accumulation of cholesterol since FXR activation leads to repression of cholesterol 7α-hydroxylase (CYP7A1) which is the rate-limiting enzyme in metabolic conversion of cholesterol to bile acids. Partial FXR agonism with lower maximum efficacy than typical FXR agonists such as 1c seems a promising concept to exploit desirable effects of FXR activation without disturbing cholesterol homeostasis (Merk, D et al. Anthranilic Acid Derivatives as Novel Ligands for Farnesoid X Receptor (FXR). Bioorg. Med. Chem. 2014, 22 (8), 2447-2460; Merk, et al. Extending the Structure—Activity Relationship of Anthranilic Acid Derivatives As Farnesoid X Receptor Modulators: Development of a Highly Potent Partial Farnesoid X Receptor Agonist. J. Med. Chem. 2014, 57 (19), 8035-8055; Merk, D. et al. Medicinal Chemistry of Farnesoid X Receptor Ligands: From Agonists and Antagonists to Modulators. Future Med. Chem. 2012, 4 (8), 1015-1036).

The soluble epoxide hydrolase (sEH) is a downstream enzyme of the CYP pathway of arachidonic acid metabolism and also holds promise in the treatment of NAFLD/NASH and other metabolic diseases such as type 2 diabetes mellitus (Shen, H. C.; Hammock, B. D. Discovery of Inhibitors of Soluble Epoxide Hydrolase: A Target with Multiple Potential Therapeutic Indications. J. Med. Chem. 2012, 55 (5), 1789-1808; Newman, J. W. et al; Epoxide Hydrolases: Their Roles and Interactions with Lipid Metabolism. Prog. Lipid Res. 2005, 44 (1), 1-51; Imig, J. D. Epoxides and Soluble Epoxide Hydrolase in Cardiovascular Physiology. Physiol. Rev. 2012, 92 (1), 101-130; Huang, H.; Weng, J.; Wang, M.-H. EETs/sEH in Diabetes and Obesity-Induced Cardiovascular Diseases. Prostaglandins Other Lipid Mediat. 2016, 125, 80-89.). It converts epoxyeicosatrienoic acids (EETs) formed by CYP enzymes from arachidonic acid to the respective dihydroxyeicosatrienoic acids (DHETs). Since EETs exhibit robust anti-inflammatory activities, sEH inhibition constitutes an anti-inflammatory strategy. sEH is expressed throughout the body with especially high levels in heart, liver and kidney. Recent results from mouse models for NASH have revealed that sEH knockout or inhibition reduces hepatic fat accumulation and hepatic inflammation under high fat diet.

NASH is associated with various risk factors such as type 2 diabetes or obesity and possesses diverse manifestations including steatosis, hepatic inflammation and fibrosis. Accordingly, several experimental strategies have revealed a therapeutic efficacy in NASH (Sanyal, A. J. Novel Therapeutic Targets for Steatohepatitis. Clin. Res. Hepatol. Gastroenterol. 2015, 39 Suppl 1, 846-50; Milic, S, et al. Nonalcoholic Steatohepatitis: Emerging Targeted Therapies to Optimize Treatment Options. Drug Des. Devel. Ther. 2015, 9, 4835-4845.) and it seems reasonable to face this multifactorial disease with more than one therapeutic mechanism treating its distinct pathological factors. However, the resulting polypharmacy with a multitude of drugs is disadvantageous suffering e.g. from complex and problematic drug-drug interactions and additional side-effects. Multi-target agents addressing the multitude of the desired therapeutic mechanisms can avoid many drawbacks of poly-pharmacology.

In light of the clinical efficacy of FXR activation in reducing NASH progression and lipid accumulation as well as the hepatic anti-inflammatory and anti-steatotic effects of sEH inhibition, dual modulation of FXR and sEH seems a promising strategy to treat NAFLD/NASH that might generate synergistic effects. Therefore, the object of the present invention is to provide dual modulators with partial agonistic activity on FXR and inhibitory potency on sEH.

The above problem is solved by a compound having the formula I:

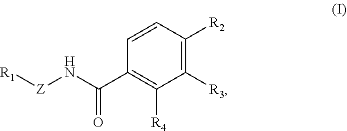

wherein R₁, R₂, R₃ and R₄ are independently selected from H, an unsubstituted, monosubstituted, or polysubstituted C₁-C₁₈ alkyl or heteroalkyl, wherein said alkyl is straight, branched or cyclic, a unsubstituted, monosubstituted or polysubstituted C₁-C₁₈ alkenyl or heteroalkenyl, wherein said alkenyl is straight, branched or cyclic, an unsubstituted, monosubstituted, or polysubstituted aryl or heteroaryl, an unsubstituted, monosubstituted, or polysubstituted benzyl group, an acyl group, such as formyl, acetyl, trichloroacetyl, fumaryl, maleyl, succinyl, benzoyl, or acyl groups being branched, heteroatom-substituted or aryl-substituted, a sugar or another acetal, and a sulfonyl group, and/or wherein R₂, R₃ and/or R₄ form together a nonsubstituted, monosubstituted, or polysubstituted ring, preferably an aromatic ring,
Z is C with or without any substitution, preferably substituted with H or alkyl;
or an isomer, racemate, prodrug, or derivative thereof, or a pharmaceutically acceptable salt or solvate of these compounds.

In some preferred embodiments R₂ is C₁-C₂₀ alkyl, preferably a branched alky, more preferably the group —C(CH₃)₃, preferably R₃ is H, —OH or OMe, and preferably R₄ is H, —OH or —OMe.

The compound is in another preferred embodiment the above defined group of compounds excluding the herein disclosed compounds 4a, 4b, 6, 7, 9, 14, 16, 19, 29, 33, 36, 42, and 45. In this regard, further preferred is any single one of the other (excluding the above) compounds disclosed in tables 1 to 8 in the example section of this application. In some embodiments the compounds of table 8 are preferred.

In one preferred embodiment the R₃ and R₄ in the above compound is H.

In some embodiments is R1 a group having the formula II:

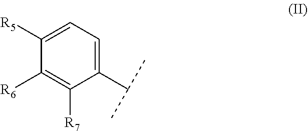

Wherein R₅, R₆ and R₇ are independently selected from H, an unsubstituted, monosubstituted, or polysubstituted C₁-C₁₈ alkyl or heteroalkyl, wherein said alkyl is straight, branched or cyclic, a unsubstituted, monosubstituted or polysubstituted C₁-C₁₈ alkenyl or heteroalkenyl, wherein said alkenyl is straight, branched or cyclic, an unsubstituted, monosubstituted, or polysubstituted aryl or heteroaryl, an unsubstituted, monosubstituted, or polysubstituted benzyl group, an acyl group, such as formyl, acetyl, trichloroacetyl, fumaryl, maleyl, succinyl, benzoyl, or acyl groups being branched, heteroatom-substituted or aryl-substituted, a sugar or another acetal, and an unsubstituted, monosubstituted, or polysubstituted amide or sulfonyl group.

Preferably R₆ and R₇ are H or halogen, preferably a halogen selected from F or Cl. Most preferably R₆ is H, and R₇ is H, F or Cl.

Preferably R₅ is a side chain of any length comprising a carboxylic acid or a suitable carboxylic acid replacement such as a typical bioisoster including but not limited to a tetrazole, a sulfonamide, an amide (such as an organic amide, a sulfonamide, or a phosphoramide) or the like.

The term “bioisoster” as used herein relates to a chemical moiety, which replaces another moiety in a molecule of an active compound without significant influence on its biological activity. Other properties of the active compound, such as for example its stability as a medicament, can be affected in this way.

As bioisoster moieties for carboxy (COOH) group can be mentioned especially 5-membered heterocyclic groups having from 1 to 4 heteroatoms selected from nitrogen, oxygen and sulphur, such as for example 1,3,4-oxadiazolyl, 1,2,3-oxadiazolyl, 1,2,5-oxadiazolyl, 1,2,4-oxadiazolyl, 1,3,4-thiadiazolyl, 1,2,4-thiadiazolyl, 1,2,3-thiadiazolyl, 1,2,5-thiadiazolyl, furyl, thienyl, pyrrolyl, pyrazolyl, imidazolyl, isoxazolyl, isothiazolyl, and N-substituted tetrazolyl. 5-Membered heterocyclic groups can be optionally substituted with 1 or 2 substituents selected from the group comprising phenyl, pyridinyl, straight or branched alkyl group, amino group, hydroxy group, fluoro, chloro, bromo, iodo, trifluoromethyl, trifluoromethoxy, trifluorothiomethoxy, alkoxy, and thioalkoxy.

As bioisoster moieties for carboxy (COOH) group can be also mentioned phenyl and 6-membered heterocyclic groups having from 1 to 4 heteroatoms selected from nitrogen, oxygen and sulphur, such as for example pyridinyl, pyrazinyl, pyridazinyl, pyrimidinyl, triazinyl, tetrazinyl, and others. Phenyl and 6-membered heterocyclic groups can be optionally substituted with 1 or 2 substituents selected from the group comprising phenyl, pyridinyl, straight or branched alkyl group, amino group, hydroxy group, fluoro, chloro, bromo, iodo, trifluoromethyl, trifluoromethoxy, trifluorothiomethoxy, alkoxy, and thioalkoxy.

In some embodiments of the invention R₁, is selected from any of the following substituents:

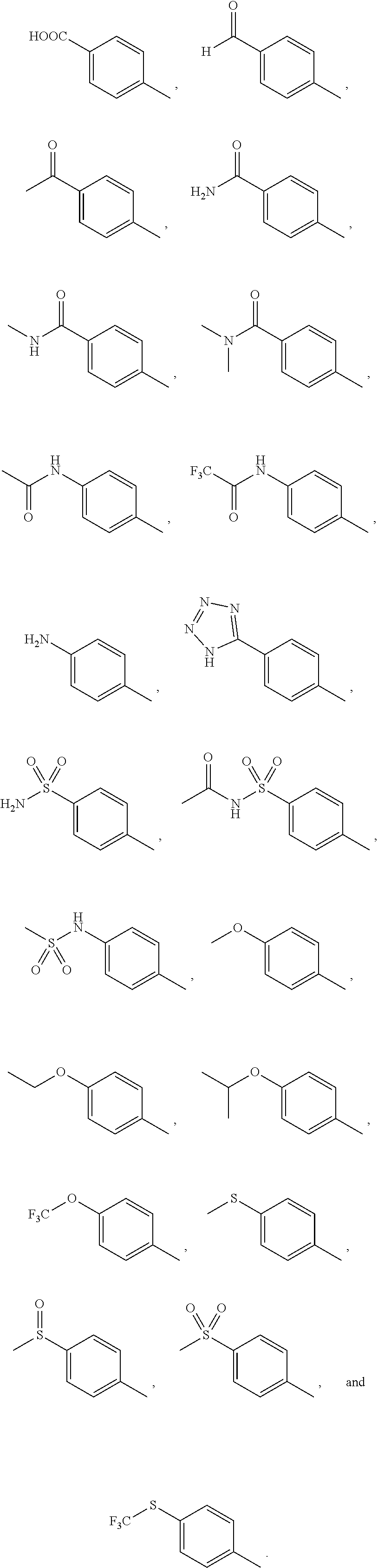

Most preferably the compound of the invention has the above formula I, wherein R₁ is selected from the group consisting of:

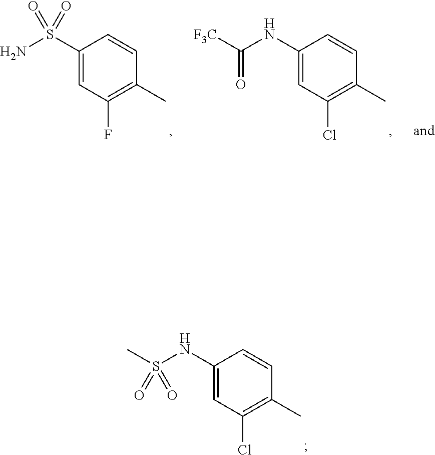

wherein Z is C, R₂ is —C(CH₃)₃, and R₄, R₃ is H.

In another embodiment, further preferred is the above compound having formula I and wherein R₁, is selected from the group consisting of

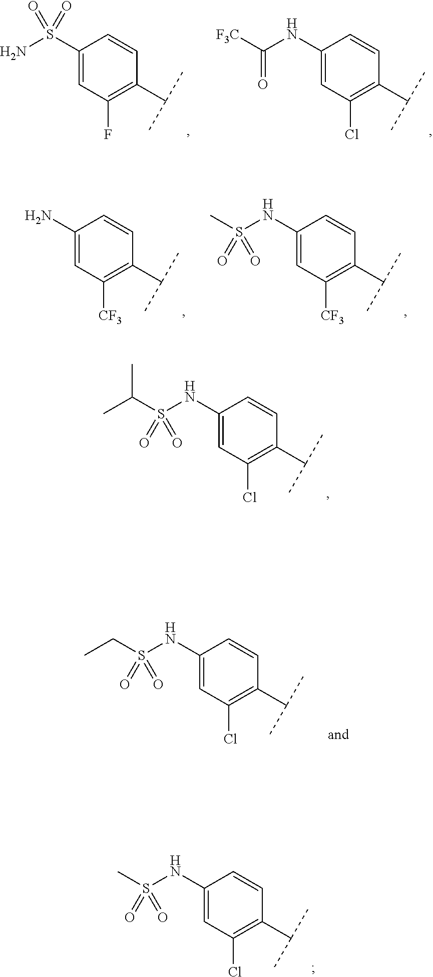

wherein Z is C, R₃ is H or OH, and R₄ is H or OH, in particular R and R₄ are not both OH; and wherein R₂ is selected from —C(CH₃)₃, —N(CH₃)₂, or the R₂ is any of the following

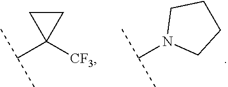

The compound according to any one of claims 1 to 5, which is a farnesoid X receptor (FXR) agonist and a soluble epoxide hydrolase (sEH) inhibitor.

Salts with a pharmaceutically unacceptable anion likewise form part of the scope of the invention as useful intermediates for the preparation or purification of pharmaceutically acceptable salts and/or for use in nontherapeutic, for example in vitro, applications. The compounds of the invention may also exist in various polymorphous forms, for example as amorphous and crystalline polymorphous forms. All polymorphous forms of the inventive compounds are within the scope of the invention and are a further aspect of the invention.

As used herein, the term “farnesoid X receptor” or “FXR” refers to all mammalian forms of such receptor including, for example, alternative splice isoforms and naturally occurring isoforms (see e.g. R. M. Huber et al., Gene 2002, 290, 35). Representative FXR species include, without limitation the rat (GenBank Accession No. NM_21745), mouse (Genbank Accession No. NM_09108), and human (GenBank Accession No. NM_05123) forms of the receptor.

The term “soluble epoxide hydrolase (sEH)” is meant to refer to a bifunctional enzyme that in humans is encoded by the EPHX2 gene (HGNC:3402). sEH is a member of the epoxide hydrolase family. This enzyme, found in both the cytosol and peroxisomes, binds to specific epoxides and converts them to the corresponding diols.

In another aspect the invention also provides a method for synthesizing the herein disclosed novel compounds.

The compounds of the invention are in particular useful in a method of treating a disease in a subject. Preferably the disease to be treated according to the invention is a disorder associated with FXR and sEH.

Also provided is, thus, a method for concomitant modulation of FXR and sEH, the method comprising the step of administering to a subject or a cell a dual FXR and she modulator as described herein before.

Another aspect then relates to a method of treating a disease in subject, the method comprising a step of administering to the subject a therapeutically effective amount of the compound of the invention, or of the pharmaceutical composition of the invention.

In context of the present disclosure the term “subject” preferably pertains to a mammal, preferably a mouse, rat, donkey, horse, cat, dog, guinea pig, monkey, ape, or preferably to a human patient, for example a patient suffering from the herein described disorders.

In preferred embodiments the disease or disorder is a metabolic disorder, preferably a metabolic disorder caused by or associated with a high-fat diet.

Liver disease is a type of damage to or disease of the liver. There are more than a hundred kinds of liver disease. The most widely spread are as follows: Fascioliasis; Hepatitis; Alcoholic liver disease; Fatty liver disease; Cirrhosis; liver; biliary; sclerosing cholangitis; Centrilobular necrosis; Budd-Chiari syndrome; Hereditary liver diseases (hemochromatosis, involving accumulation of iron in the body, and Wilson's disease); transthyretin-related hereditary amyloidosis; and Gilbert's syndrome.

As used herein, the term “liver disease” refers to any disease or disorder that affects the liver. Examples of liver disease include, but are not limited to, Alagille Syndrome; Alcohol-Related Liver Disease; Alpha-1 Antitrypsin Deficiency; Autoimmune Hepatitis; Benign Liver Tumors; Biliary Atresia; Cirrhosis; Galactosemia; Gilbert Syndrome; Hemochromatosis; Hepatitis A; Hepatitis B; Hepatitis C; Hepatocellular Carcinoma; Hepatic Encephalopathy; Liver Cysts; Liver Cancer; Newborn Jaundice; Non-Alcoholic Fatty Liver Disease (including nonalcoholic fatty liver and nonalcoholic steatohepatitis); Primary Biliary Cirrhosis (PBC); Primary Sclerosing Cholangitis (PSC); Reye Syndrome; Type I Glycogen Storage Disease and Wilson Disease.

The terms “Non-alcoholic fatty liver” or “Non-alcoholic fatty liver disease” (NAFLD) refers to a condition which is one cause of a fatty liver, occurring when fat is deposited in the liver not due to excessive alcohol use. NAFLD is related to insulin resistance and the metabolic syndrome and may respond to treatments originally developed for other insulin-resistant states (e.g. diabetes mellitus type 2) such as weight loss, metformin and thiazolidinediones. NAFLD can be subclassified as non-alcoholic steatohepatitis (NASH) and nonalcoholic fatty liver (NAFL). NASH is the more extreme form of NAFLD, and is regarded as a major cause of cirrhosis of the liver of unknown cause.

Most patients with NAFLD have few or no symptoms. Patients may complain of fatigue, malaise, and dull right-upper-quadrant abdominal discomfort. Mild jaundice may be noticed although this is rare. More commonly NAFLD is diagnosed following abnormal liver function tests during routine blood tests. NAFLD is associated with insulin resistance and metabolic syndrome (obesity, combined hyperlipidemia, diabetes mellitus (type II) and high blood pressure). Common findings are elevated liver enzymes and a liver ultrasound showing steatosis. An ultrasound may also be used to exclude gallstone problems (cholelithiasis). A liver biopsy (tissue examination) is the only test widely accepted as definitively distinguishing NASH from other forms of liver disease and can be used to assess the severity of the inflammation and resultant fibrosis.

Nonalcoholic steatohepatitis (NASH) is a common, often “silent” liver disease. The major feature in NASH is fat in the liver, along with inflammation and damage. Most people with NASH feel well and are not aware that they have a liver problem. Nevertheless, NASH can be severe and can lead to cirrhosis, in which the liver is permanently damaged and scarred and no longer able to work properly.

NASH is usually first suspected in a person who is found to have elevations in liver tests that are included in routine blood test panels, such as alanine aminotransferase (ALT) or aspartate aminotransferase (AST). When further evaluation shows no apparent reason for liver disease and when x rays or imaging studies of the liver show fat, NASH is suspected. The only means of providing a definitive diagnosis of NASH and separating it from simple fatty liver is a liver biopsy. NASH is diagnosed when fat along with inflammation and damage to liver cells is observed from the biopsy. If the tissue shows fat without inflammation and damage, NAFL or NAFLD is diagnosed. Currently, no blood tests or scans can reliably provide this information.

Therefore, preferred diseases to be treated with the compounds of the invention are liver diseases, such as non-alcoholic fatty liver disease or non-alcoholic steatohepatitis (NASH).

As used herein, the term “therapeutically effective amount” means that amount of a compound that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder.

In yet another aspect there is provided a pharmaceutical composition comprising a compound of the invention together with a pharmaceutically acceptable carrier and/or excipient.

The compound(s) of the invention can also be administered in combination with further active ingredients. The amount of a compound of the formula I required to achieve the desired biological effect depends on a number of factors, for example the specific compound chosen, the intended use, the mode of administration and the clinical condition of the patient.

The daily dose is generally in the range from 0.3 mg to 100 mg (typically from 3 mg to 50 mg) per day per kilogram of body weight, for example 3-10 mg/kg/day. An intravenous dose may be, for example, in the range from 0.3 mg to 1.0 mg/kg, which can suitably be administered as infusion of 10 ng to 100 ng per kilogram of body weight per minute. Suitable infusion solutions for these purposes may contain, for example, 0.1 ng to 100 mg, typically 1 ng to 100 mg, per milliliter. Single doses may contain, for example, 1 mg to 10 g of the active ingredient. Thus, ampoules for injections may contain, for example, from 1 mg to 100 mg, and orally administrable single-dose formulations, for example tablets or capsules, may contain, for example, from 1.0 to 1000 mg, typically from 10 to 600 mg. For treatment of the abovementioned conditions, the compounds of the formula I themselves may be used as the compound, but they are preferably present with a compatible carrier in the form of a pharmaceutical composition. The carrier must of course be acceptable in the sense that it is compatible with the other constituents of the composition and is not harmful to the patient's health. The carrier may be a solid or a liquid or both and is preferably formulated with the compound as a single dose, for example as a tablet, which may contain from 0.05% to 95% by weight of the active ingredient. Other pharmaceutically active substances may likewise be present, including other compounds of formula I. The inventive pharmaceutical compositions can be produced by one of the known pharmaceutical methods, which essentially involve mixing the ingredients with pharmacologically acceptable carriers and/or excipients.

Inventive pharmaceutical compositions are those suitable for oral, rectal, topical, peroral (for example sublingual) and parenteral (for example subcutaneous, intramuscular, intradermal or intravenous) administration, although the most suitable mode of administration depends in each individual case on the nature and severity of the condition to be treated and on the nature of the compound of formula I used in each case. Coated formulations or medicament forms are also within the scope of the invention. Sugar-coated formulations and sugar-coated slow-release formulations are also within the scope of the invention. Preference is given to acid- and gastric juice-resistant formulations. Suitable gastric juice-resistant coatings comprise cellulose acetate phthalate, polyvinyl acetate phthalate, hydroxypropylmethylcellulose phthalate and anionic polymers of methacrylic acid and methyl methacrylate. Suitable pharmaceutical compounds for oral administration may be in the form of separate, i.e. single-dose, units, for example capsules, cachets, lozenges, film tablets, sugar-coated tablets, soluble tablets, sublingual tablets, oral tablets or tablets, each of which contains a defined amount of the compound of formula I; as powders or granules; as solution or suspension in an aqueous or nonaqueous liquid; or as an oil-in-water or water-in-oil emulsion. These compositions may, as already mentioned, be prepared by any suitable pharmaceutical method which includes a step in which the active ingredient and the carrier (which may consist of one or more additional ingredients) are brought into contact. The compositions are generally produced by uniform and homogeneous mixing of the active ingredient with a liquid and/or finely divided solid carrier, after which the product is shaped if necessary. For example, a tablet can be produced by compressing or molding a powder or granules of the compound, where appropriate with one or more additional ingredients. Compressed tablets can be produced by tableting the compound in free-flowing form such as, for example, a powder or granules, where appropriate mixed with a binder, glidant, inert diluent (filler) and/or one (or more) surfactant(s)/dispersant(s) in a suitable machine. Molded tablets or granules can be produced by molding the pulverulent compound moistened with an inert liquid diluent in a suitable machine.

Pharmaceutical compositions suitable for peroral (sublingual) administration include lozenges which contain a compound of formula I with a flavoring, typically sucrose, and gum arabic or tragacanth, and pastilles which comprise the compound in an inert base such as gelatin and glycerol or sucrose and gum arabic.

Pharmaceutical compositions suitable for parenteral administration comprise preferably sterile aqueous preparations of a compound of formula I, which are preferably isotonic with the blood of the intended recipient. These preparations are preferably administered intravenously, although administration may also take place by subcutaneous, intramuscular or intradermal injection. These preparations can preferably be produced by mixing the compound with water and making the resulting solution sterile by a suitable sterilization process (e.g. steam sterilization, sterile filtration) and isotonic with blood. Injectable compositions of the invention generally contain from 0.1 to 5% by weight of the active compound. Pharmaceutical compositions suitable for rectal administration are preferably in the form of single-dose suppositories. These can be produced by mixing a compound of formula I with one or more conventional solid carriers, for example cocoa butter, and shaping the resulting mixture.

Pharmaceutical compositions suitable for topical use on the skin are preferably in the form of ointment, cream, powder, lotion, paste, spray, aerosol or oil. Carriers which can be used are petrolatum, lanolin, polyethylene glycols, alcohols and combinations of two or more of these substances. The active ingredient is generally present in a concentration of 0.1 to 15% by weight of the composition, for example 0.5 to 2%.

Transdermal administration is also possible. Pharmaceutical compositions suitable for transdermal uses may be in the form of single patches which are suitable for long-term close contact with the patient's epidermis. Such patches suitably contain the active ingredient in an aqueous solution which is buffered where appropriate, dissolved and/or dispersed in an adhesive or dispersed in a polymer. A suitable active ingredient concentration is about 1% to 35%, preferably about 3% to 15%. A particular option is for the active ingredient to be released by electrotransport or iontophoresis as described, for example, in Pharmaceutical Research, 2(6): 318 (1986).

The present invention will now be further described in the following examples with reference to the accompanying figures and sequences, nevertheless, without being limited thereto. For the purposes of the present invention, all references as cited herein are incorporated by reference in their entireties. In the Figures:

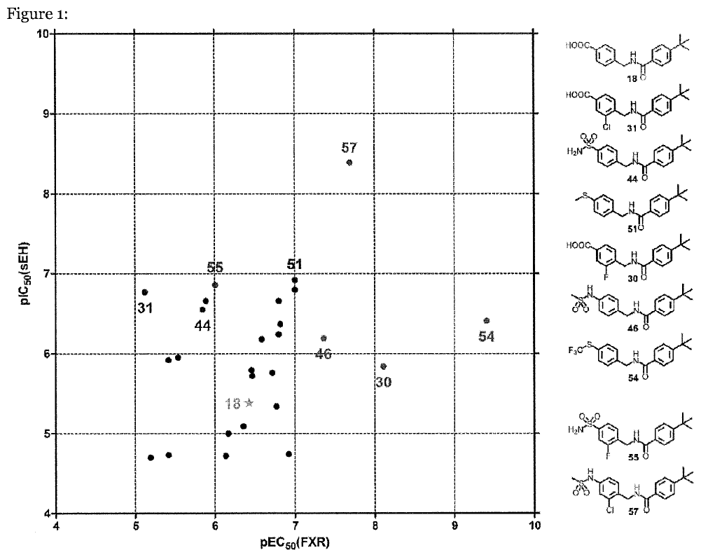

FIG. 1 A potency plot of the dual modulators indicated 30, 46 and 54 (red) as most potent partial FXR agonists while 31, 44 and 51 (blue) emerged for highest sEH inhibitory potency. Consequently, the structural features of these compounds were combined to increase the dual activity resulting in 55 and 57 (green, table 7).

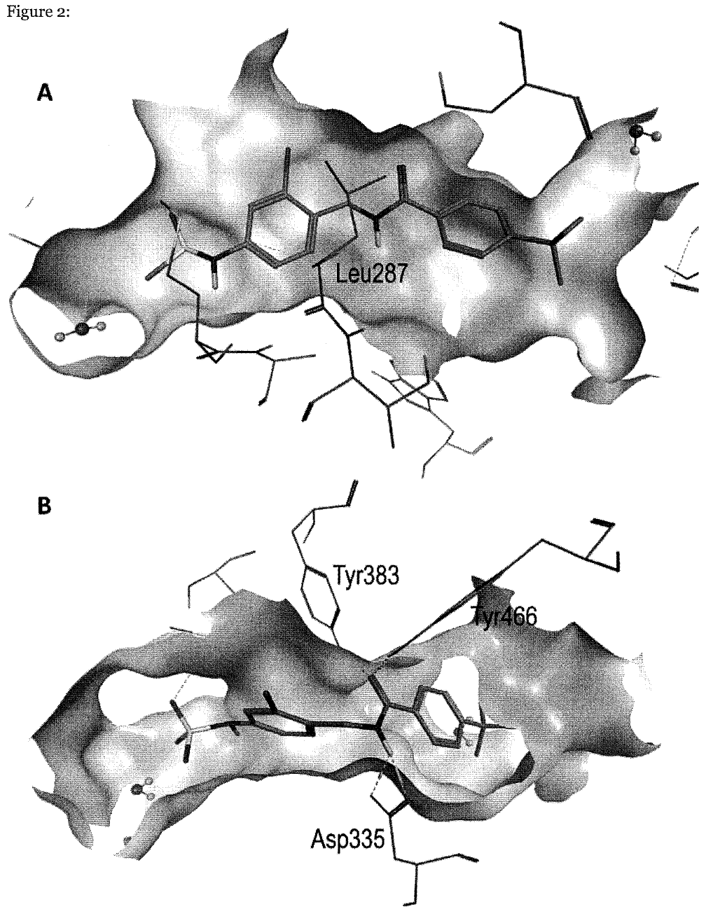

FIG. 2 Molecular docking of 57: (A) Binding mode of compound 57 in the FXR-LBD (PDB code 4QE8). (B) Binding mode of compound 57 in the sEH (PDB code 3I28). Molecular surface is colored by lipophilicity (green: lipophilic; magenta: hydrophilic). Selected side chains are shown as lines, compound 57 is displayed as sticks.

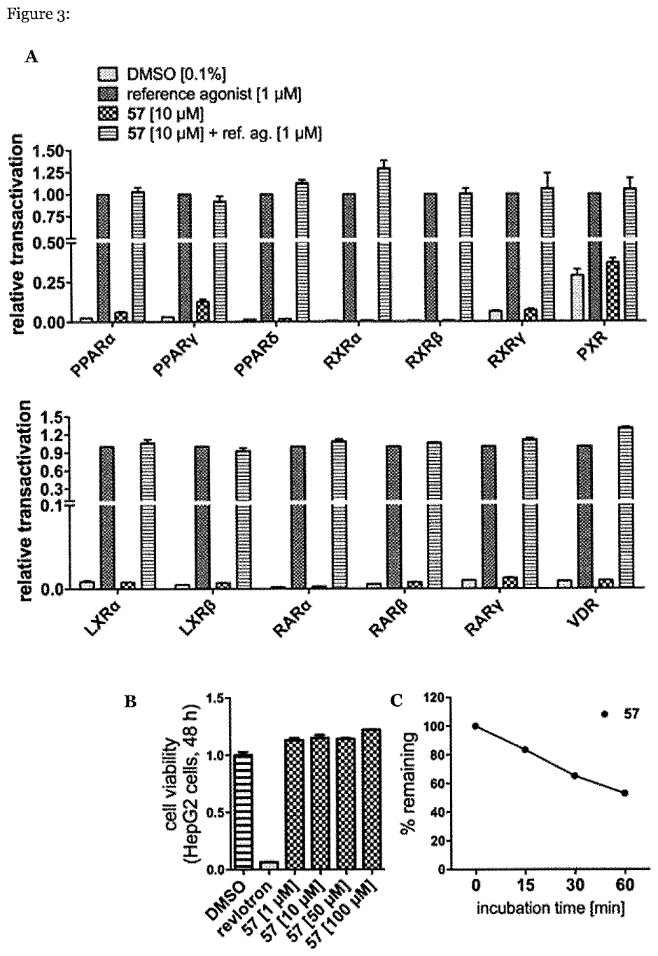

FIG. 3: In vitro characterization of 57: (A) Selectivity profile: except for a moderate activity on PPARγ (EC₅₀=14.7±0.9 μM, 30±1% max.), 57 was inactive on related nuclear receptors and, therefore highly selective for FXR (values are mean±SEM; n=3). (B) 57 revealed no cytotoxicity in a common WST-1 assay on HepG2 cells up to 100 μM (values are mean±SEM; n=4). (C) 57 displayed an acceptable in vitro microsomal stability with more than 50% remaining after 60 minutes incubation with Wistar rat liver microsomes (values are mean±SEM; n=3).

FIG. 4: Determination of cellular activity of 57: (A) FXR target gene mRNA quantification in HepG2 cells after 8 or 16 hours incubation with 57 at 0.1 μM and 1 μM compared to CDCA (1b) at 50 μM: 57 caused a concentration-independent partial induction of BSEP, SHP, PPARα, GLUT4, FGF19, PDK4 and FABP1 as well as a concentration-independent partial repression of CYP7A1, SREBP1c and FAS (vehicle treated control cells are defined as 100%; values are mean±SEM; n=4). (B) 57 did not cause significant modulation of PPARγ target genes CD36 and FAM3A (pioglitazone (PIO) as positive control; values are mean±SEM; n=3). (C) Soluble epoxide hydrolase activity in cell homogenates from HepG2 cells: Dual modulator 57 inhibits conversion of 14.15-EET-d11 to 14.15-DHET-d11 by cellular sEH with an IC₅₀ value of approximately to nM and exerts statistically significant inhibition at concentrations as low as 0.1 nM (values are mean±SEM; n=3). (*p<0.05; **p<0.01; ***p<0.001)

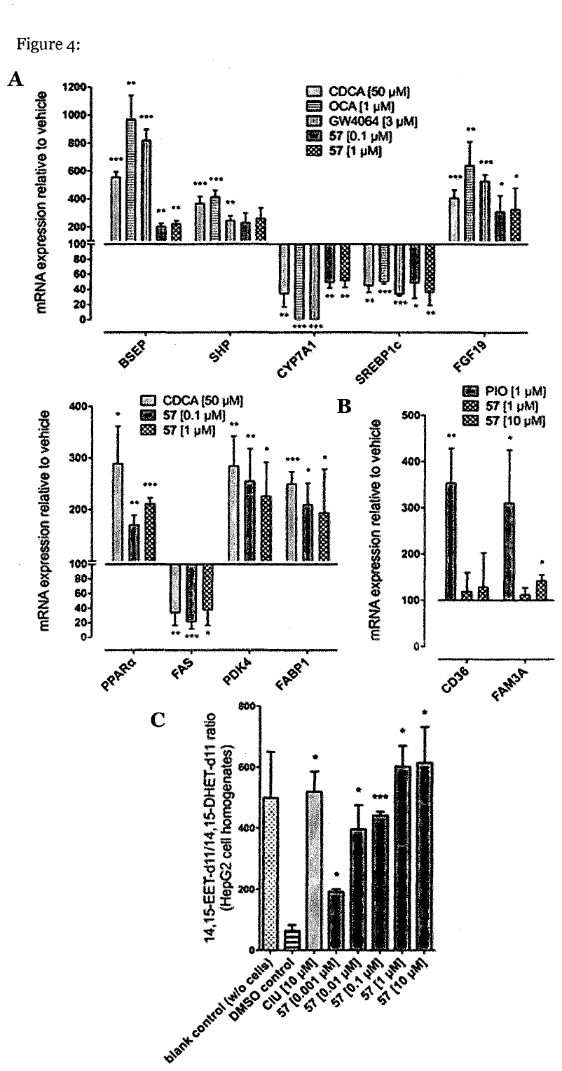

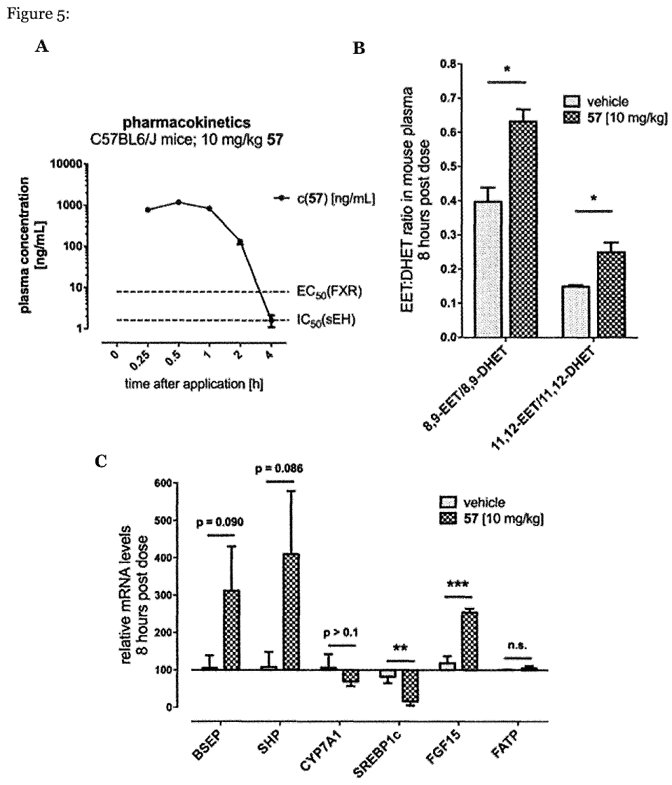

FIG. 5: In vivo characterization of 57: (A) In vivo pharmacokinetic evaluation of 57 revealed rapid uptake, high bioavailability and moderate though acceptable half-life of the dual modulator. 57 achieved effective plasma concentrations above IC₅₀ (sEH) and EC₅₀ (FXR) values for roughly 3.5 hours after a single oral dose of 10 mg/kg body weight. (B) An approximately 2-fold increase in EET/DHET ratios in mouse plasma 8 h post dose indicated that 57 inhibited sEH in vivo. (C) Quantification of mRNA levels of FXR target genes BSEP, SHP, CYP7A1, SREBP1c and FGF15 as well as PPARγ target gene FATP 8 h post dose compared to vehicle treated mice (100%). 57 showed a trend to induction of BSEP and SHP and slightly repressed CYP7A1. Moreover, 57 significantly repressed SREBP1c and significantly induced FGF15 indicating FXR modulation in vivo and potentially beneficial effects in NASH. PPARγ target gene FATP was not modulated in vivo. (n(vehicle)=3; n(57)=6; *p<0.05; **p<0.01; *p<0.001).

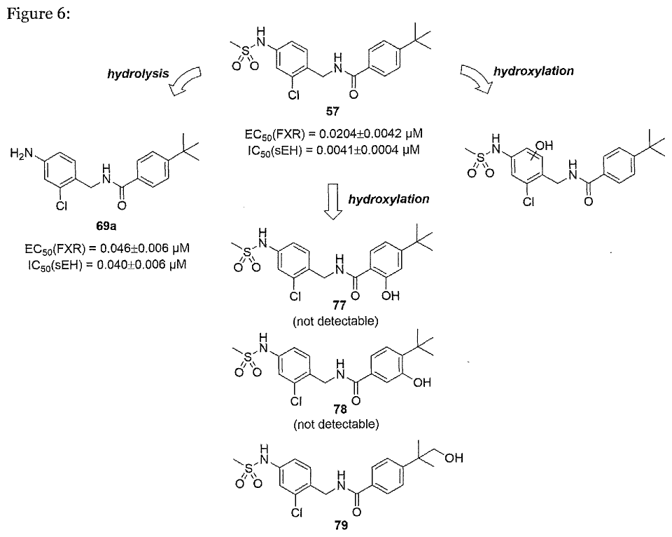

FIG. 6: (or scheme 10): In vitro metabolism of 57. Hydrolysis of the sulfonamide moiety of dual modulator 57 generates metabolite 69a (confirmed by LC-MS-MS) which displays high dual modulatory potency and can contribute to pharmacodynamics activity. LC-MS-MS analysis also indicates the presence of a metabolite resulting from hydroxylation at the tert-butylbenzamide residue Of the three possible isomers 77, 78 and 79, 77 and 78 were not detectable confirming metabolic hydroxylation on the tert-butyl group leading to 79. Furthermore, 57 is hydroxylated on the aromatic ring of the benzyl substituent.

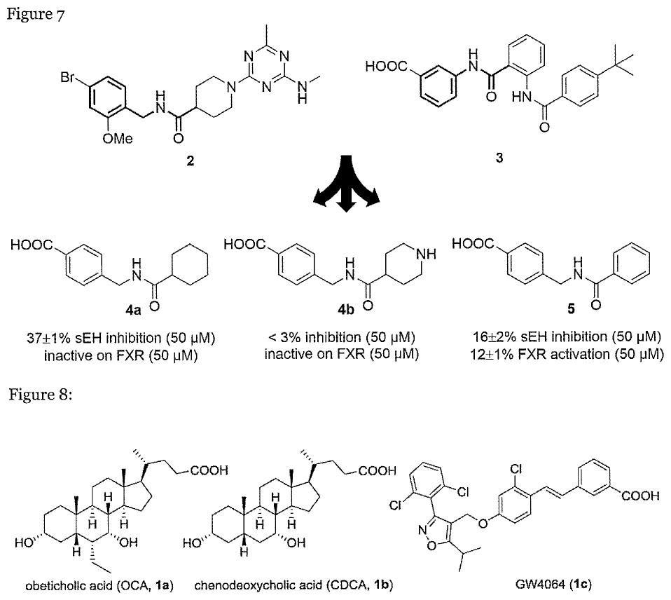

FIG. 7: (or scheme 1) shows Important FXR ligands: obeticholic acid (1a), physiological agonist CDCA (1b) and synthetic reference FXR agonist (1c).

FIG. 8: (or scheme 2) shows discovery of lead compound 5 by merging the pharmacophores of sEH inhibitor GSK2188931B (2) and partial FXR agonist 3.

### EXAMPLES

In order to develop agents with potent dual activity on FXR and sEH, the inventors initially screened representative compounds of the in-house library of FXR modulators but failed to identify a lead compound with inhibitory potency on sEH amongst them. Therefore, the inventors searched for a merged pharmacophore from known partial FXR agonists and known sEH inhibitors. Several sEH inhibitors contain an amide or urea residue mimicking the epoxide moiety cleaved by the enzyme. The sEH inhibitor GSK2188931B (2) comprising an N-benzylamide residue as pharmacophore shares some structural similarity with recently reported¹⁵ partial FXR agonists such as 3. The inventors therefore extracted the similar structural features of both compounds and combined them in the minimal dual pharmacophore 4a containing the N-benzylamide residue for sEH inhibition and a carboxylic acid group for FXR activation (FIG. 8, scheme 2). Compound 4a exhibited moderate sEH inhibition of 37±1% at 50 μM but was inactive on FXR. 4b incorporating the piperidine moiety of template 2 displayed even lower activity and was inactive on sEH and FXR. However, when the inventors replaced the saturated ring in 4a and 4b by an aromatic moiety in 5 the inventors achieved the desired dual activity with 12±1% FXR activation and 16±2% sEH inhibition at 50 μM concentration. For the low fragment-like size (MW 255 Da) of 5 the inventors considered this moderate potency sufficient and systematically investigated the structure activity relationship (SAR) of 5 as dual sEH and FXR modulator (FIG. 2).

### Example 1: Synthesis

N-benzyl benzamides 4-57 and 77-78 were prepared according to schemes 3-9. Synthesis of aminomethylbenzene precursors 58a-j started with radical bromination of the respective methylbenzene derivatives 59a-j using NBS and AIBN to bromomethylbenzenes 60a-j. Subsequently, bromomethylbenzenes 60a-j were applied to a two-step Staudinger reaction using sodium azide to generate azides 61a-j and triphenylphosphine in water for their reduction. Aminomethylbenzene derivative 58k was prepared by reduction of 4-amino-2-chlorobenzonitrile 62 using LiAlH₄. Aminomethylbenzene derivatives 58l-w were commercially available. Subsequently, 58a-w were reacted with carbonyl chlorides 63a-o in presence of pyridine or with carboxylic acids 64a-f in presence of EDC and 4-DMAP to yield compounds 18, 19, 22, 35, 36, 44, 47-51, 54, 55, 68 and 69a-c or their esters 65a-h (scheme 3). Compound 68 was treated with BrCH₂COOCH₃ to generate the ester 65i. All esters 65a-i were hydrolyzed to the final products 16, 20 and 23-32 under alkaline conditions (scheme 4). Urea 21 was prepared from 4-aminobenzoic acid (66) and 4-tert-butylphenylisocyanate (67) with NEt₃ and subsequent hydrolysis with lithium hydroxide (scheme 5). The free carboxylic acid 18 served for the preparation of amides 37-39 using ammonium chloride, methylammonium chloride or dimethylammonium chloride and EDC/DMAP. Reduction of 18 with LiAlH₄ yielded ethyl alcohol derivative 33 which was further converted to aldehyde 34 with PCC (scheme 6). The inverted amides 40, 41 and 56, the inverted sulfonamides 46 and 57 as well as N-acyl sulfonamide 45 were generated from 42, 44, and 69a with EDC/DMAP for carboxylic acid activation. Tetrazole 43 was available from nitrile 36 by cycloaddition of NaN₃ under Cu₂O catalysis (scheme 7). Oxidation of methylmercaptane 51 to sulfoxide 52 and sulfone 53 was achieved using suitable equivalents of meta-chloroperbenzoic acid (mCPBA, scheme 8) and, finally, preparation of methoxy derivatives 76a-b according to the standard procedure (scheme 3) and their demethylation with BBr₃ yielded phenolic derivatives 77 and 78 (scheme 9).

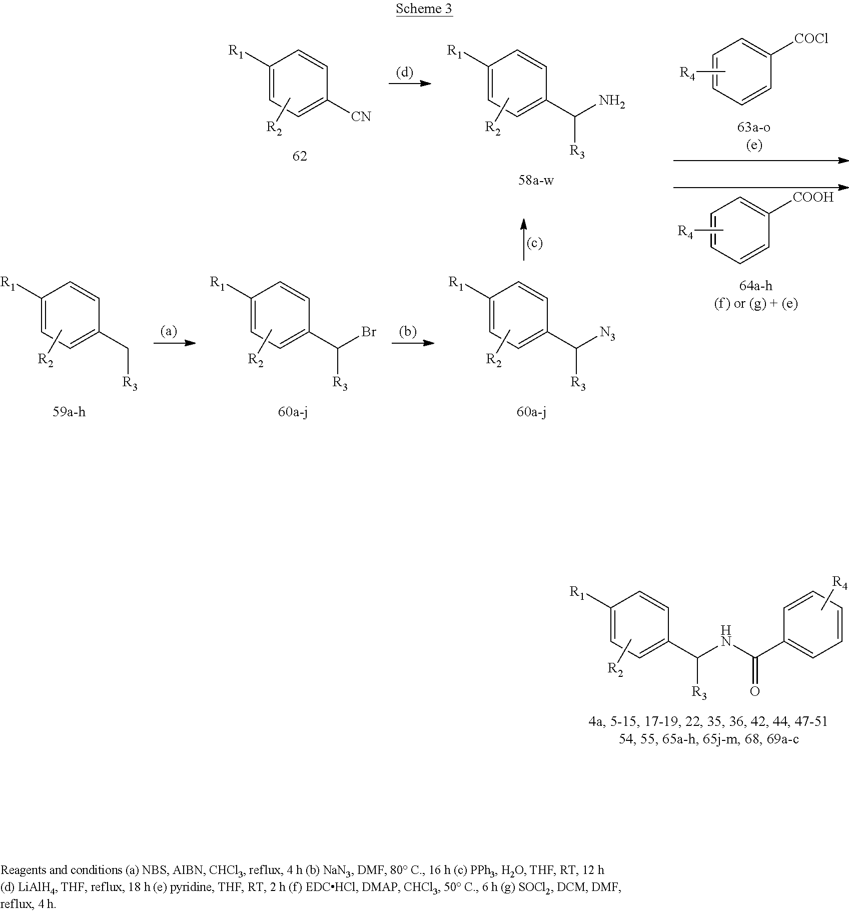

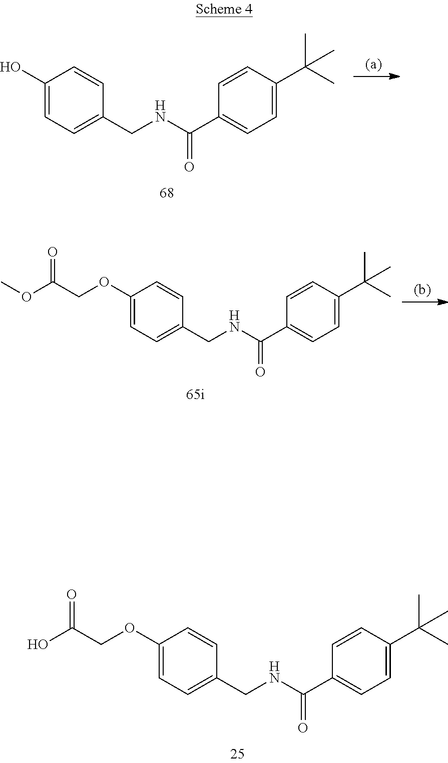

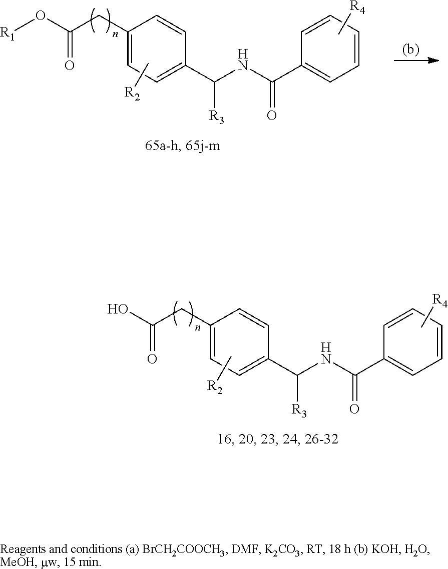

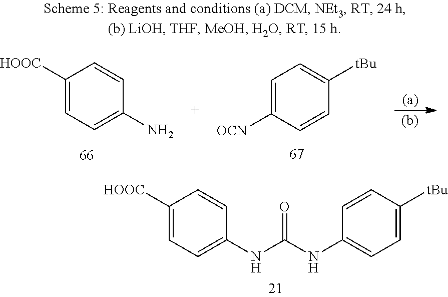

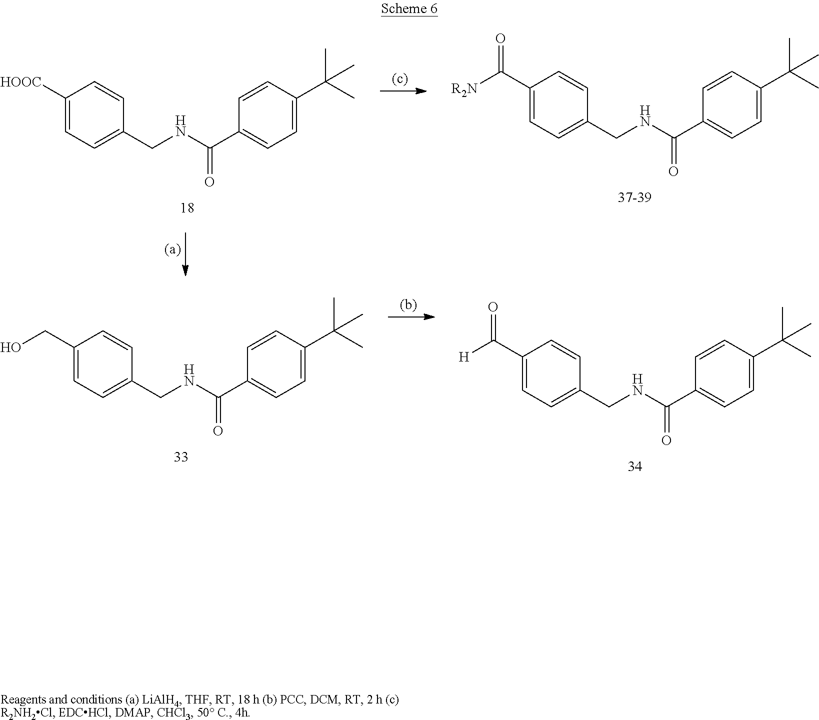

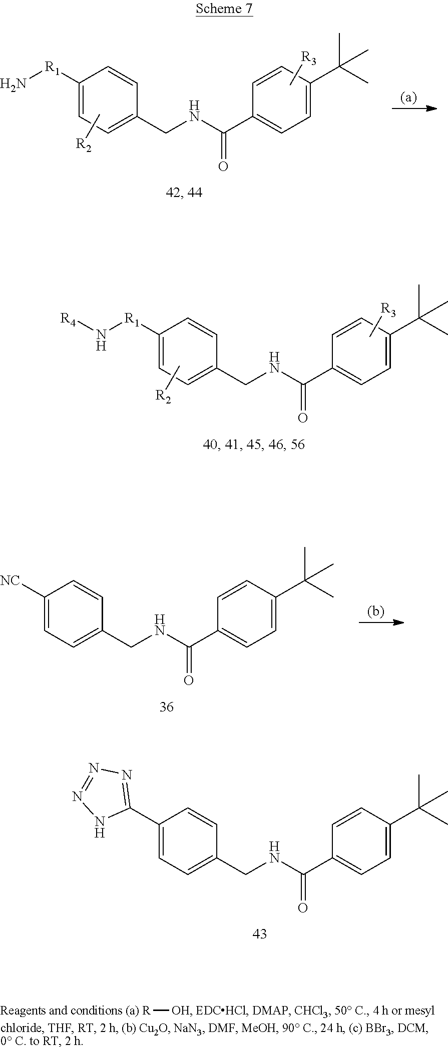

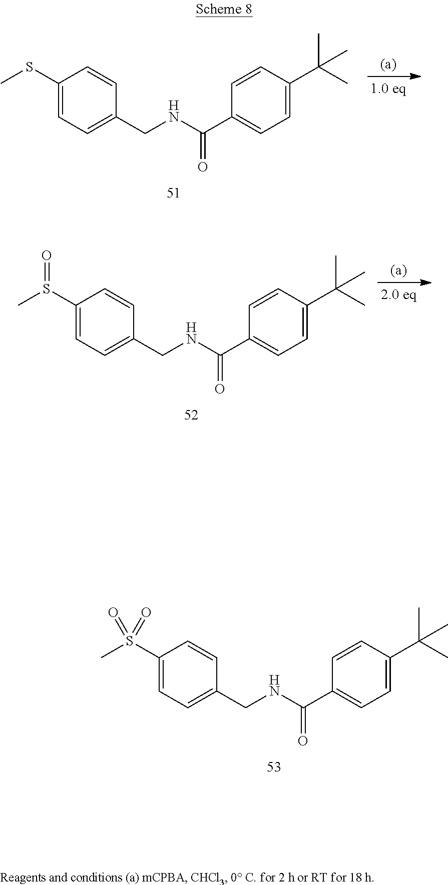

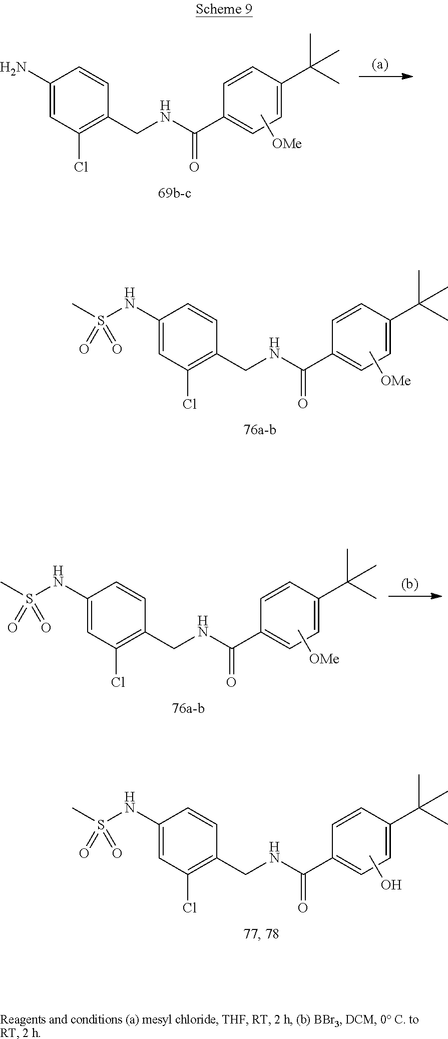

### Example 2: Biological Evaluation

To determine FXR agonistic activity, test compounds 4-57 were characterized in a full-length (fl) FXR reporter gene assay in HeLa cells. This assay is based on a reporter construct containing a firefly luciferase under the control of the FXR response element from bile salt export protein (BSEP). FXR and its hetero dimer partner retinoid X receptor (RXR) as expression constructs under the control of a CMV promoter as well as a constitutively expressed renilla luciferase (SV40 promoter) for normalization and toxicity control were co-transfected. The synthetic FXR agonist GW4064 (1c) was used as reference agonist and its transactivation activity at 3 μM was defined as 100% activation. The assay was validated with diverse known FXR agonists which yielded potencies in good agreement with literature (GW4064 (1c): EC₅₀=0.51±0.16 μM, OCA (1a): EC₅₀=0.16±0.02 μM and CDCA (1b): EC₅₀=18±1 μM). The well-characterized sEH inhibitor CIU³⁶ had no activity in this assay at 10 μM excluding unspecific effects of sEH inhibitors. The sEH inhibitory potency of the test compounds was quantified in a fluorescence-based assay using recombinant enzyme and the fluorogenic sEH substrate PHOME³⁷ that is hydrolyzed to a fluorescent 6-methoxynaphtaldehyde by sEH.

### Example 3: Structure Activity Relationship & Structural Optimization

As first step in the SAR study (table 1), the inventors introduced additional methyl groups (6-8) in every position of the benzamide partial structure of 5. While all methylated derivatives 6-8 exhibited weak sEH inhibitory activity, FXR only tolerated a 4-methyl group in 8 indicating that the FXR binding pocket offers additional space especially in this direction. A 3-methyl group (7) generated the highest sEH inhibition amongst the methylated derivatives and therefore the inventors prepared compounds (9, 10) comprising two methyl substituents in order to combine the improvements on both targets. 2,4-dimethyl derivative 9 was nearly inactive while 3,4-dimethyl derivative 10 exhibited improved dual activity although its maximum relative activation on FXR was rather low.

As the additional methyl groups did not significantly improve the potency on either target the inventors also investigated the introduction of larger residues on the benzamide moiety and characterized biphenyl derivatives 11-13. All three biphenyls 11-13 revealed considerably higher potency than the respective methylbenzamides 6-8 while the rank order of potency remained unchanged. FXR tolerated 3-substitution (12) but favored 4-substitution (13) and sEH could equally be inhibited by the 3-(12) and the 4-biphenyl derivative (13).

TABLE 1
In vitro activity of 4-13 on FXR and sEH (data represents mean ± SEM, n = 3-6)

| # | 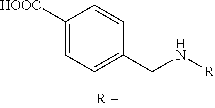 | FXR activation EC₅₀ (max. rel. activation) or % activation at indicated conc. | sEH inhibition IC₅₀ or % inhibition at indicated conc. |
|---|---|---|---|
| 4a | 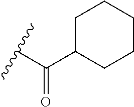 [SMILES: *C(=O)C1CCCCC1] | inactive (50 μM) | 37 ± 1% inhibition (50 μM) |
| 4b | 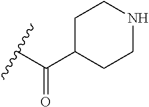 [SMILES: *C(=O)C1CCNCC1] | inactive (50 μM) | inactive (50 μM) |
| 5 | 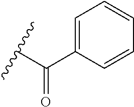 [SMILES: *C(=O)c1ccccc1] | 12 ± 1% activation (50 μM) | 16 ± 2% inhibition (50 μM) |
| 6 | 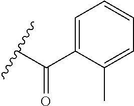 [SMILES: *C(=O)c1ccccc1C] | inactive (50 μM) | 14 ± 1% inhibition (50 μM) |
| 7 | 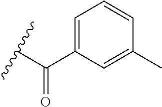 [SMILES: *C(=O)c1cccc(C)c1] | inactive (50 μM) | 37 ± 1% inhibition (50 μM) |
| 8 | 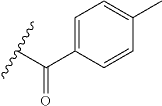 [SMILES: *C(=O)c1ccc(C)cc1] | 15 ± 3% activation (50 μM) | 24 ± 1% inhibition (50 μM) |
| 9 | 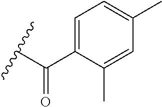 [SMILES: *C(=O)c1ccc(C)cc1C] | inactive (50 μM) | 7 ± 2% inhibition (50 μM) |
| 10 | 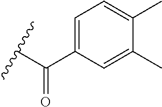 [SMILES: *C(=O)c1ccc(C)c(C)c1] | 0.32 ± 0.03 μM (12 ± 1%) | 52 ± 1% inhibition (50 μM) |
| 11 | 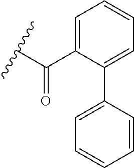 [SMILES: *C(=O)c1ccccc1-c1ccccc1] | 22 ± 1% activation (50 μM) | 19 ± 2% inhibition (50 μM) |
| 12 | 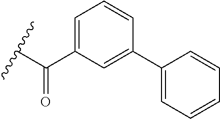 [SMILES: *C(=O)c1cccc(-c2ccccc2)c1] | 0.67 ± 0.17 μM (14 ± 1%) | 10.0 ± 1.2 μM |
| 13 | 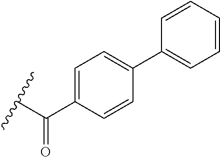 [SMILES: *C(=O)c1ccc(-c2ccccc2)cc1] | 0.44 ± 0.04 μM (24 ± 1%) | 8.1 ± 0.1 μM |

The initial SAR results suggested that 3- and 4-substitution at the benzamide residue in 5 was tolerated by both targets eventually improving the dual potency and, therefore, the inventors investigated various 3,4-disubstitutions (14-16) as well as 2-naphtyl derivative 17 (table 2). However, 14-17 failed to produce a significant improvement in the dual potency on FXR and sEH. 3,4-dichloro (14) and 3,4-bis(trifluoromethyl) substitution (16) were only tolerated by sEH and 2-naphtyl derivative 17 was less active on FXR than 10. Only 3,4-dimethoxybenzamide 15 was favorable since it improved the maximum relative activation on FXR without affecting the EC₅₀ value and weakly inhibited sEH activity.

TABLE 2
In vitro activity of 14-17 on FXR and sEH (data represents mean ± SEM, n = 3-6)

| # | 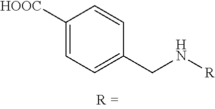 | FXR activation EC₅₀ (max. rel. activation) or % activation at indicated conc. | sEH inhibition IC₅₀ or % inhibition at indicated conc. |
|---|---|---|---|
| 10 | 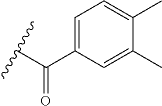 [SMILES: *C(=O)c1ccc(C)c(C)c1] | 0.32 ± 0.03 μM (12 ± 1%) | 52 ± 1% inhibition (50 μM) |
| 14 | 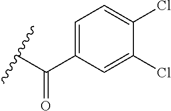 [SMILES: *C(=O)c1ccc(Cl)c(Cl)c1] | inactive (50 μM) | 10.5 ± 0.3 μM |
| 15 | 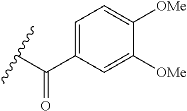 [SMILES: *C(=O)c1ccc(OC)c(OC)c1] | 0.37 ± 0.02 μM (18 ± 1%) | 26 ± 2% inhibition (50 μM) |
| 16 | 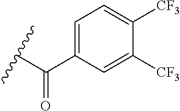 [SMILES: *C(=O)c1ccc(C(F)(F)F)c(C(F)(F)F)c1] | inactive (50 μM) | 55 ± 1% inhibition (50 μM) |
| 17 | 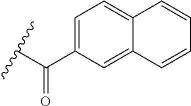 [SMILES: *C(=O)c1ccc2ccccc2c1] | 1.36 ± 0.08 μM (18 ± 1%) | 45 ± 1% inhibition (50 μM) |

So far, 4-biphenyl derivative 13 revealed the highest dual potency indicating that both targets tolerated bulky 4-substituents at the benzamide residue (table 3). Introduction of a 4-tert-butyl moiety in 18 led to further improvement of the dual activity while the more polar 4-dimethylaminobenzamide 19 as tert-butyl mimic was considerably less active. Combination of the favorable bulky tert-butyl residue of 18 and the 3-methoxy group of 15 in 20 did not generate additive effects and, therefore, the inventors selected 4-tert-butyl derivative 18 for further optimization.

TABLE 3
In vitro activity of 18-20 on FXR and sEH (data represents mean ± SEM, n = 3-6)

| # | 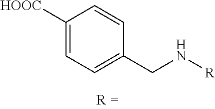 | FXR activation EC₅₀ (max. rel. activation) or % activation at indicated conc. | sEH inhibition IC₅₀ or % inhibition at indicated conc. |
|---|---|---|---|
| 8 | 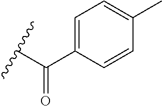 [SMILES: *C(=O)c1ccc(C)cc1] | 15 ± 3% activation (50 μM) | 24 ± 1% inhibition (50 μM) |
| 13 | 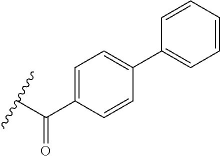 [SMILES: *C(=O)c1ccc(-c2ccccc2)cc1] | 0.44 ± 0.04 μM (24 ± 1%) | 8.1 ± 0.1 μM |
| 18 | 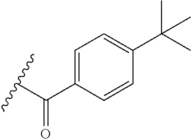 [SMILES: *C(=O)c1ccc(C(C)(C)C)cc1] | 0.37 ± 0.04 μM (13 ± 1%) | 4.2 ± 0.8 μM |
| 19 | 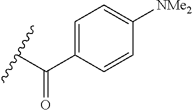 [SMILES: *C(=O)c1ccc(N(C)C)cc1] | inactive (50 μM) | 19 ± 1% inhibition (50 μM) |
| 20 | 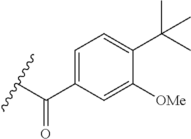 [SMILES: *C(=O)c1ccc(C(C)(C)C)c(OC)c1] | 1.2 ± 0.4 μM (23 ± 1%) | 5.04 ± 0.30 μM |

With 18 as new lead the inventors then focused on optimization of the N-benzyl substituent (table 4). Exchanging the N-benzylbenzamide structure of 18 by a diphenylurea in 21 as classical sEH pharmacophore was poorly tolerated by both targets. Similarly, shifting the carboxylic acid moiety from 4-position in 18 to 3-position in 22 caused a marked decline in potency. Variation of the side chain length from benzoic acid (18) to phenylacetic acid (23) diminished potency on both targets while phenylpropionic acid 24 exerted about equal activity as 18. This SAR might eventually explained by a water-mediated interaction of 18 and displacement of the water by 24 as observed in previous studies.¹⁵ Phenoxyacetic acid 25 exhibited similar activity on FXR as phenylpropionic acid 24 but the ether residue was poorly tolerated by sEH and significantly diminished inhibitory potency by about a factor 10.

TABLE 4
In vitro activity of 21-25 on FXR and sEH (data represents mean ± SEM, n = 3-6)

| # | 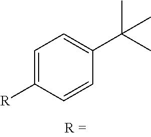 | FXR activation EC₅₀ (max. rel. activation) or % activation at indicated conc. | sEH inhibition IC₅₀ or % inhibition at indicated conc. |
|---|---|---|---|
| 18 | 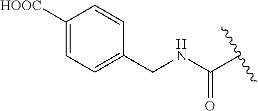 [SMILES: *C(=O)NCc1ccc(C(=O)O)cc1] | 0.37 ± 0.04 μM (13 ± 1%) | 4.2 ± 0.8 μM |
| 21 | 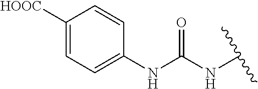 [SMILES: *NC(=O)Nc1ccc(C(=O)O)cc1] | 3.8 ± 1.7 μM (14 ± 1%) | 18.5 ± 2.8 μM |
| 22 | 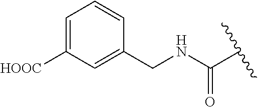 [SMILES: *C(=O)NCc1cccc(C(=O)O)c1] | 1.70 ± 0.03 μM (14 ± 1%) | 26 ± 2% inhibition (50 μM) |
| 23 | 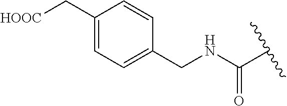 [SMILES: *C(=O)NCc1ccc(CC(=O)O)cc1] | 6.4 ± 1.2 μM (19 ± 1%) | 20.0 ± 5.1 μM |
| 24 | 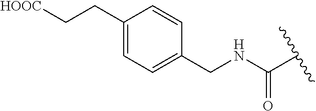 [SMILES: *C(=O)NCc1ccc(CCC(=O)O)cc1] | 0.35 ± 0.03 μM (16 ± 1%) | 1.63 ± 0.02 μM |
| 25 | 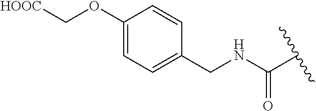 [SMILES: *C(=O)NCc1ccc(OCC(=O)O)cc1] | 0.73 ± 0.18 μM (21 ± 1%) | 19.0 ± 0.7 μM |

As no significant improvement in dual potency was achieved by variations in the molecular geometry and distance between pharmacophoric features (21-25) the inventors then explored the possibility of introducing additional substituents at the benzoic acid aromatic ring of 18 (table 5). Substituents in 2-position (26-28) remarkably increased potency on FXR with 2-chloro derivative 28 as highly potent partial FXR agonist. However, sEH inhibitory activity simultaneously dropped significantly. In contrast, 3-substitution (29-31) with increasing substituent size was beneficial for inhibitory potency on sEH with 3-chlorobenzoic acid 31 as highly potent sEH inhibitor. On FXR, 3-methyl substitution (29) entirely abolished activity, while 3-chloro derivative 31 was still active but with considerably lower potency. 3-flourobenzoic acid 30 was highly potent on FXR. Methylation in benzylic position (32) significantly enhanced agonistic activity on FXR but, not surprisingly, it was not tolerated by sEH. As the amide moiety mimics the epoxide of EETs and an attacking water molecule in the enzyme's active site³⁴, steric hindrance in benzylic position remarkably diminishes inhibitory potency on sEH. Altogether, several additional residues on the benzoic acid residue improved the activity on FXR or sEH but no position could be identified where further substitution generated enhanced and balanced dual potency.

TABLE 5
In vitro activity of 26-32 on FXR and sEH (data represents mean ± SEM, n = 3-6)

| # | 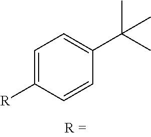 | FXR activation EC₅₀ (max. rel. activation) or % activation at indicated conc. | sEH inhibition IC₅₀ or % inhibition at indicated conc. |
|---|---|---|---|
| 18 | 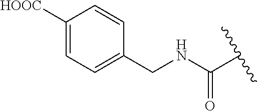 [SMILES: *C(=O)NCc1ccc(C(=O)O)cc1] | 0.37 ± 0.04 μM (13 ± 1%) | 4.2 ± 0.8 μM |
| 26 | 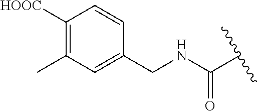 [SMILES: *C(=O)NCc1ccc(C(=O)O)c(C)c1] | 0.16 ± 0.05 μM (14 ± 1%) | 53 ± 1% inhibition (50 μM) |
| 27 | 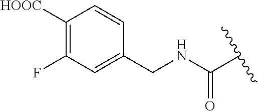 [SMILES: *C(=O)NCc1ccc(C(=O)O)c(F)c1] | 0.14 ± 0.06 μM (28 ± 2%) | 20.3 ± 1.2 μM |
| 28 | 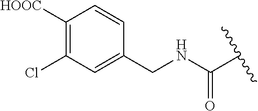 [SMILES: *C(=O)NCc1ccc(C(=O)O)c(Cl)c1] | 0.0088 ± 0.0033 μM (21 ± 2%) | 50 ± 1% inhibition (50 μM) |
| 29 | 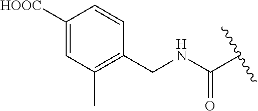 [SMILES: *C(=O)NCc1ccc(C(=O)O)cc1C] | inactive (50 μM) | 0.68 ± 0.06 μM |
| 30 | 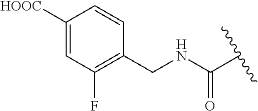 [SMILES: *C(=O)NCc1ccc(C(=O)O)cc1F] | 0.0078 ± 0.0007 μM (26 ± 1%) | 1.43 ± 0.10 μM |
| 31 | 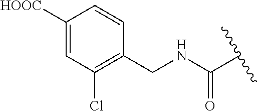 [SMILES: *C(=O)NCc1ccc(C(=O)O)cc1Cl] | 7.5 ± 0.4 μM (25 ± 1%) | 0.17 ± 0.01 μM |
| 32 | 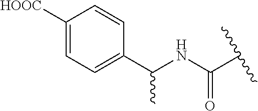 [SMILES: *C(=O)NC(C)c1ccc(C(=O)O)cc1] | 0.076 ± 0.027 μM (24 ± 1%) | 37 ± 1% inhibition (50 μM) |

Therefore, the inventors investigated the SAR of the carboxylic acid in 18 and introduced several alternative polar residues and bioisosters (table 6). Alcohol 33 was equally potent as 18 on sEH but inactive on FXR whereas aldehyde 34 still activated FXR with about 10-fold lower potency but was significantly more potent on sEH compared to 18. Methylketone 35 showed remarkably improved activity on both targets and was the first dual modulator with nanomolar potency on FXR and sEH. This SAR indicated that a carbonyl moiety but not an alcohol instead of the carboxylic acid was sufficient for FXR activation and that sEH preferred less polar groups in this position. The reduced potency of aldehyde 34 might be due to low stability in the cellular context of the flFXR assay. Nitrile 36 inhibited sEH with nanomolar potency but was inactive on FXR. The amides 37-39 were only moderately potent sEH inhibitors which again indicated that more polar residues in this position were disadvantageous. On FXR, the amides 37-39 significantly gained in potency with increasing substitution on the nitrogen atom. Primary amide 37 was considerably less active than carboxylic acid 18 while N-methylamide 38 comprised equal potency as 18. Another methyl group in N,N-dimethylamide 39 further enhanced the potency. Inversion of the amide in N-acetylaniline 40 reduced FXR agonistic activity but introduction of three fluorine atoms in N-trifluoroacetylaniline 41 was very favorable and generated a balanced nanomolar dual modulator. Free aniline 42 still inhibited sEH but was inactive on FXR. Tetrazole 43 as classical bioisoster of the carboxylic acid possessed slightly enhanced potency on FXR accompanied by reduced sEH inhibitory activity compared to 18. The considerably less acidic sulfonamide 44 somewhat inverted the activity profile of 18 and was more potent on sEH than on FXR. In order to increase the acidity of 44, the inventors prepared N-acetylsulfonamide 45 but it was inactive on FXR and remarkably lost potency on sEH compared to 44. Inversion of the sulfonamide residue in 46 had a similar effect as inversion of the amide in 40 and also yielded a potent dual modulator.

Finally, the inventors exchanged the carboxylic acid of 18 by a methoxy group in 47 which surprisingly resulted in considerable potency on both targets. Ethoxy derivative 48 revealed equal potency on FXR and sEH while isopropyloxy analogue 49 was equally active on FXR but could not be characterized on sEH due to insolubility. A slight improvement in potency on sEH was achieved with trifluoromethoxy derivative 50 which revealed high and well-balanced dual potency. Replacement of oxygen by sulfur in methylmercaptane 51 generated an even more potent dual modulator with half-maximal activity on both targets at approximately 0.1 μM. When mercaptane 51 was oxidized to sulfoxide 52 or sulfone 53, potency on FXR dropped remarkably while 52 was still quite active on sEH and only 53 revealed significantly diminished inhibitory potency. In case of the mercaptane as carboxylic acid replacement, introduction of a trifluoromethyl group in 54 led to a marked increase in potency on FXR and generated a sub-nanomolar partial agonist. However, trifluoromethylmercaptane 54 revealed slightly diminished activity on sEH compared to methylmercaptane 51 and, therefore comprised a high but poorly balanced dual potency.

TABLE 6
In vitro activity of 33-54 on FXR and sEH (data represents mean ± SEM, n = 3-6)

| # | 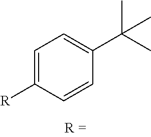 | FXR activation EC₅₀ (max. rel. activation) or % activation at indicated conc. | sEH inhibition IC₅₀ or % inhibition at indicated conc. |
|---|---|---|---|
| 18 | 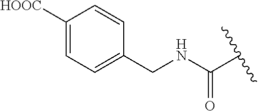 [SMILES: *C(=O)NCc1ccc(C(=O)O)cc1] | 0.37 ± 0.04 μM (13 ± 1%) | 4.2 ± 0.8 μM |
| 33 | 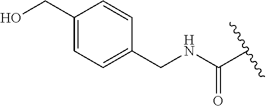 [SMILES: *C(=O)NCc1ccc(CO)cc1] | inactive (50 μM) | 2.05 ± 31 μM |
| 34 | 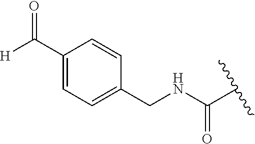 [SMILES: *C(=O)NCc1ccc(C=O)cc1] | 5.3 ± 1.5 μM (22 ± 2%) | 0.51 ± 0.04 μM |
| 35 | 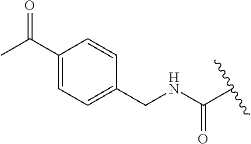 [SMILES: *C(=O)NCc1ccc(C(C)=O)cc1] | 0.10 ± 0.01 μM (29 ± 1%) | 0.157 ± 0.009 μM |
| 36 | 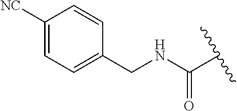 [SMILES: *C(=O)NCc1ccc(C#N)cc1] | inactive (50 μM) | 0.91 ± 0.07 μM |
| 37 | 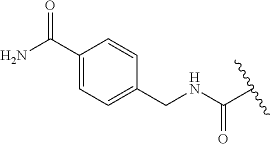 [SMILES: *C(=O)NCc1ccc(C(N)=O)cc1] | 3.8 ± 0.3 μM (27 ± 1%) | 1.2 ± 0.2 μM |
| 38 | 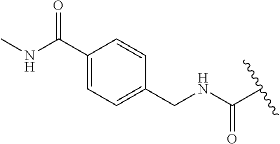 [SMILES: *C(=O)NCc1ccc(C(=O)NC)cc1] | 0.34 ± 0.03 μM (29 ± 1%) | 1.9 ± 0.3 μM |
| 39 | 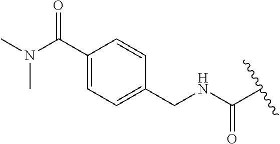 [SMILES: *C(=O)NCc1ccc(C(=O)N(C)C)cc1] | 0.17 ± 0.01 μM (17 ± 1%) | 4.6 ± 0.5 μM |
| 40 | 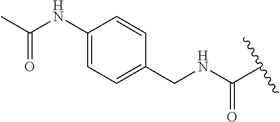 [SMILES: *C(=O)NCc1ccc(NC(C)=O)cc1] | 2.9 ± 0.2 μM (31 ± 1%) | 1.12 ± 0.02 μM |
| 41 | 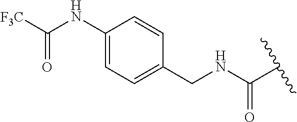 [SMILES: *C(=O)NCc1ccc(NC(=O)C(F)(F)F)cc1] | 0.26 ± 0.11 μM (20 ± 1%) | 0.66 ± 0.01 μM |
| 42 | 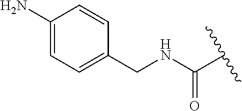 [SMILES: *C(=O)NCc1ccc(N)cc1] | inactive (50 μM) | 3.5 ± 0.2 μM |
| 43 | 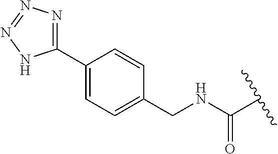 [SMILES: *C(=O)NCc1ccc(-c2nnn[nH]2)cc1] | 0.12 ± 0.02 μM (23 ± 1%) | 18.2 ± 3.2 μM |
| 44 | 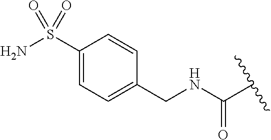 [SMILES: *C(=O)NCc1ccc(S(N)(=O)=O)cc1] | 1.4 ± 0.2 μM (16 ± 1%) | 0.28 ± 0.12 μM |
| 45 | 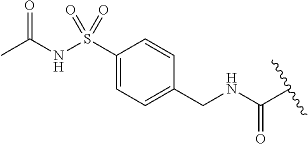 [SMILES: *C(=O)NCc1ccc(S(=O)(=O)NC(C)=O)cc1] | inactive (50 μM) | 12.7 ± 0.4 μM |
| 46 | 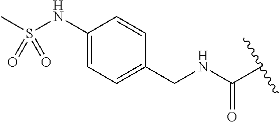 [SMILES: *C(=O)NCc1ccc(NS(C)(=O)=O)cc1] | 0.044 ± 0.009 μM (22 ± 1%) | 0.65 ± 0.04 μM |
| 47 | 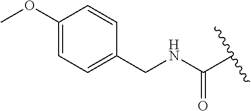 [SMILES: *C(=O)NCc1ccc(OC)cc1] | 0.15 ± 0.04 μM (19 ± 1%) | 0.43 ± 0.01 μM |
| 48 | 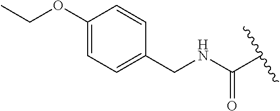 [SMILES: *C(=O)NCc1ccc(OCC)cc1] | 0.16 ± 0.01 μM (31 ± 1%) | 0.57 ± 0.05 μM |
| 49 | 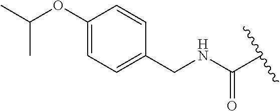 [SMILES: *C(=O)NCc1ccc(OC(C)C)cc1] | 0.17 ± 0.01 μM (26 ± 1%) | n.d. (poor solubility) |
| 50 | 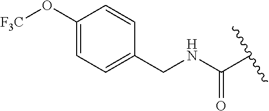 [SMILES: *C(=O)NCc1ccc(OC(F)(F)F)cc1] | 0.16 ± 0.04 μM (26 ± 1%) | 0.22 ± 0.06 μM |
| 51 | 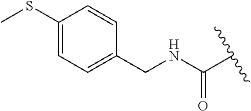 [SMILES: *C(=O)NCc1ccc(SC)cc1] | 0.10 ± 0.01 μM (33 ± 1%) | 0.12 ± 0.01 μM |
| 52 | 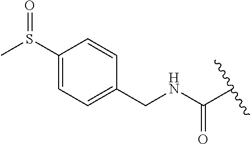 [SMILES: *C(=O)NCc1ccc(S(C)=O)cc1] | 1.3 ± 0.4 μM (24 ± 1%) | 0.22 ± 0.03 μM |
| 53 | 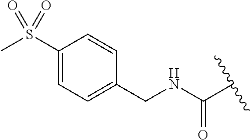 [SMILES: *C(=O)NCc1ccc(S(C)(=O)=O)cc1] | 3.8 ± 0.5 μM (34 ± 1%) | 1.2 ± 0.1 μM |
| 54 | 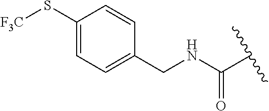 [SMILES: *C(=O)NCc1ccc(SC(F)(F)F)cc1] | 0.00040 ± 0.00004 μM (19 ± 1%) | 0.39 ± 0.05 μM |

Since neither single modification of our optimized lead compound 18 alone was able to improve potency on both targets to low nanomolar values, the inventors evaluated the possibility of combining the most favorable structural alterations for each target in one molecule. To identify derivatives with outstanding activity on one of the targets, the inventors plotted pIC₅₀ (sEH) versus pEC₅₀ (FXR) values of the most potent compounds (FIG. 1). This potency plot revealed a 3-fluorosubstitution at the benzyl moiety (30), the trifluoromethylmercaptane residue (54) and the inverted sulfonamide 46 as very favorable on FXR. Since trifluoroacetamide 41 structurally resembled 46 and also possessed high potency on FXR, it was additionally selected for combination. Concerning sEH inhibition, 3-chlorosubstitution at the benzyl moiety (31), the methylmercaptane residue (51) and sulfonamide 44 protruded from the other derivatives. 51 and 54 were omitted for recombination for their proportionally poor solubility and because the two moieties could not be merged. Instead, 30, 31, 41, 44 and 46 were selected for structural recombination (table 7).

Introduction of the 3-fluorine atom of 30 in sulfonamide 44 yielded the dual modulator 55 that revealed the anticipated increase in FXR potency. However, this improvement was merely moderate and did not produce the desired dual modulator with low nanomolar potency. In contrast, merging of 31 and 41 in N-(3-chlorophenyl)trifluoroacetamide 56 and combination of 31 and 46 in N-(3-chlorophenyl)methanesulfonamide 57 was accompanied by a remarkable rise in potency on both targets. With respective EC₅₀ values of 14±1 nM and 20.4±4.2 nM for partial FXR activation as well as IC₅₀ values of 8.9±1.6 nM and 4.1±0.4 nM for sEH inhibition, the dual modulators 56 and 57 finally comprised the desired low nanomolar potency on both targets. Amongst these two dual modulators, 57 revealed significantly higher aqueous solubility (1.5 μg/mL) than 56 (<0.1 μg/mL (LLOQ)) and was, therefore, selected for further in vitro evaluation. Isothermal titration calorimetry (ITC, supporting figure S7) revealed a Kd of 0.13 μM for 57 and indicated enthalpic binding (ΔH=−19.5 kcal/mol, ΔS=−34.0 cal/mol·K). The discrepancy between cellular EC₅₀ value and Kd seems due to absence of coactivators in the ITC experiment that might significantly affect the binding equilibrium. The high binding energy might partly arise from potentially marked conformational changes in the FXR-LBD upon binding

TABLE 7
In vitro activity of 55-57 on FXR and sEH (data represents mean ± SEM, n = 3-6)

| # | 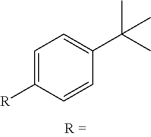 | FXR activation EC₅₀ (max. rel. activation) or % activation at indicated conc. | sEH inhibition IC₅₀ or % inhibition at indicated conc. |
|---|---|---|---|
| 55 | 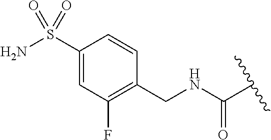 [SMILES: *C(=O)NCc1ccc(S(N)(=O)=O)cc1F] | 0.97 ± 0.11 μM (34 ± 1%) | 0.137 ± 0.018 μM |
| 56 | 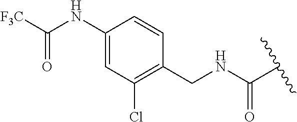 [SMILES: *C(=O)NCc1ccc(NC(=O)C(F)(F)F)cc1Cl] | 0.014 ± 0.001 μM (21 ± 1%) | 0.0089 ± 0.0016 μM |
| 57 | 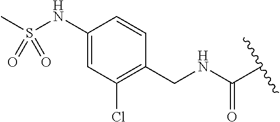 [SMILES: *C(=O)NCc1ccc(NS(C)(=O)=O)cc1Cl] | 0.0204 ± 0.0042 μM (35 ± 1%) | 0.0041 ± 0.0004 μM |

Further, the following additional compounds were produced and tested for their activity towards FXR activation and she inhibition (see table 8).

TABLE 8
In vitro activity of additional compounds on FXR and sEH (data represents
mean ± SEM, n = 3-6)

| | | FXR activation EC50 (max. rel. activation) or % activation at indicated conc. | sEH inhibition IC50 or % inhibition at indicated conc. |
|---|---|---|---|
| 78 | 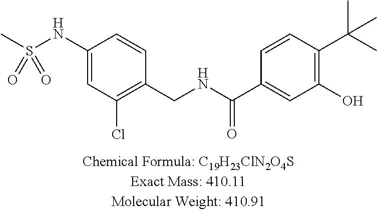 [SMILES: CC(C)(C)c1ccc(C(=O)NCc2ccc(NS(C)(=O)=O)cc2Cl)cc1O] | 0.003 ± 0.000001 μM (24 ± 1%) | 0.0160 ± 0.0006 μM |
| 77 | 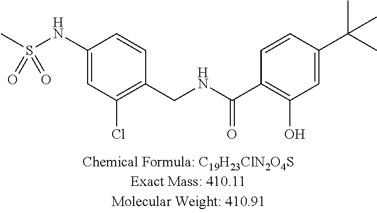 [SMILES: CC(C)(C)c1ccc(C(=O)NCc2ccc(NS(C)(=O)=O)cc2Cl)c(O)c1] | antagonistic | 0.38 ± 0.05 μM |
| 69a | 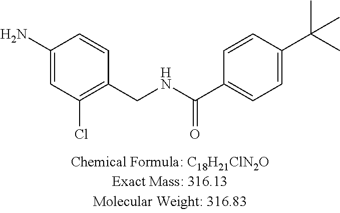 [SMILES: CC(C)(C)c1ccc(C(=O)NCc2ccc(N)cc2Cl)cc1] | 0.045 ± 0.011 μM (29 ± 1%) | 0.040 ± 0.006 μM |
| 80 | 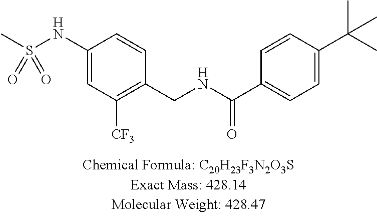 [SMILES: CC(C)(C)c1ccc(C(=O)NCc2ccc(NS(C)(=O)=O)cc2C(F)(F)F)cc1] | 1.2 ± 0.18 μM (19 ± 1%) | 0.002 ± 0.00008 μM |
| 81 | 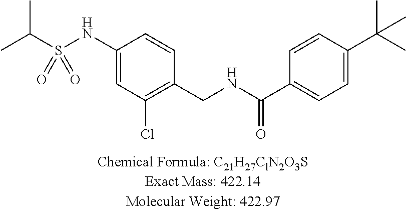 [SMILES: CC(C)S(=O)(=O)Nc1ccc(CNC(=O)c2ccc(C(C)(C)C)cc2)c(Cl)c1] | 0.42 ± 0.02 μM (11 ± 1%) | 0.011 ± 0.0003 μM |
| 82 | 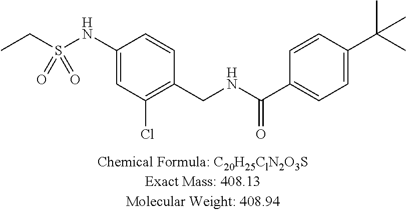 [SMILES: CCS(=O)(=O)Nc1ccc(CNC(=O)c2ccc(C(C)(C)C)cc2)c(Cl)c1] | 0.49 ± 0.05 μM (23 ± 1%) | 0.006 ± 0.0005 μM |
| 83 | 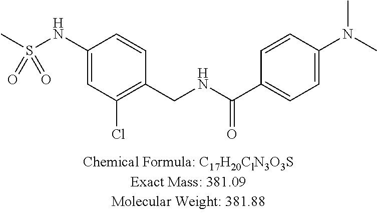 | 1.46 ± 0.023 μM (23 ± 1%) | 0.045 ± 0.002 μM |
| 84 | 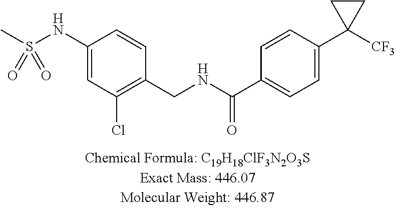 [SMILES: CS(=O)(=O)Nc1ccc(CNC(=O)c2ccc(C3(C(F)(F)F)CC3)cc2)c(Cl)c1] | 0.54 ± 0.02 μM (31 ± 1%) | 0.004 ± 0.0004 μM |
| 85 | 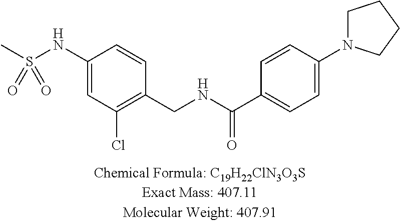 [SMILES: CS(=O)(=O)Nc1ccc(CNC(=O)c2ccc(N3CCCC3)cc2)c(Cl)c1] | 0.25 ± 0.012 μM (27 ± 1%) | 0.041 ± 0.004 μM |

The following procedures were used to synthesize the compounds:

### 1-(4-Amino-1-chlorophenyl)methanamine (58k)

LiAlH₄ (1 M in THF, 16.4 mL, 16.4 mmol, 2.5 eq) was cooled to 0° C. 4-Amino-2-chlorobenzonitrile 62 (1.0 g, 6.6 mmol, 1.0 eq) in 3 mL THF was slowly added to the mixture. After evolution of H₂ had ceased, the mixture was allowed to warm to room temperature and then refluxed for 16 h. After cooling to room temperature, the mixture was diluted with 10 mL THF and then cooled to 0° C. 1 mL 10% NaOH solution and 1.8 mL water were added dropwise. The colorless precipitate was filtered through celite and washed with 15 mL diethyl ether. Evaporation of the organic solvents from the filtrate yielded 58k as yellow oil (0.77 g, 75%). ¹H NMR (500 MHz, DMSO-d₆) δ=7.11 (d, J=8.2 Hz, 1H), 6.58 (d, J=2.2 Hz, 1H), 6.48 (dd, J=8.2, 2.2 Hz, 1H), 5.19 (s, 2H), 3.59 (s, 2H). ¹³C NMR (126 MHz, DMSO-d₆) δ=148.56, 132.33, 129.67, 127.74, 113.63, 112.72, 42.88.

### N-(4-Amino-2-chlorobenzyl)-4-(tert-butyl)benzamide (69a)

1-(4-Amino-2-chlorophenyl)methanamine 58k (0.31 g, 2.0 mmol, 1.1 eq) was dissolved in 10 mL CHCl₃, 5 mL NEt₃ were added and the mixture was cooled to 0° C. 4-tert-Butylbenzoyl chloride 630 (0.35 mL, 1.8 mmol, 1.0 eq) was slowly added over 10 min and the mixture was stirred for two hours at room temperature. Then, 50 mL 10% aqueous hydrochloric acid were added, phases were separated and the aqueous layer was washed with 30 mL EtOAc. The aqueous layer was brought to pH 10 with Na₂CO₃ solution and extracted three times with 80 mL EtOAc at a time. The combined organic layers were dried over Na₂SO₄ and the solvent was evaporated in vacuum. Further purification was performed by column chromatography with petrolether/EtOAc (90:10) as mobile phase to obtain 69a as yellow solid (0.57 g, 97%). R_{f}(petrolether/EtOAc=67:33)=0.26. ¹H NMR (500 MHz, MeOH-d₄) δ=7.81 (dt, J=8.6, 2.3 Hz, 2H), 7.53 (dt, J=8.6, 2.3 Hz, 2H), 7.45 (d, J=8.3 Hz, 1H), 7.31 (d, J=2.2 Hz, 1H), 7.15 (dd, J=8.3, 2.2 Hz, 1H), 4.64 (s, 2H), 1.36 (s, 9H). ¹³C NMR (126 MHz, MeOH-d₄) δ=170.32, 156.67, 140.89, 135.23, 132.33, 131.39, 128.29, 126.59, 122.96, 120.82, 119.16, 42.13, 35.36, 31.54. HRMS (MALDI): m/z calculated 317.14152 for C₁₈H₂₁ClN₂O, found 317.14130 [M+H]⁺.

### N-(4-Amino-2-chlorobenz-4-(tert-buyl)-4-(tert-butyl-2-methoxy)benzamide (69b)

4-Amino-2-chlorobenzamine (58k, 0.23 g, 0.15 mmol, 1.3 eq) was dissolved in 30 mL dry CHCl₃. 4-DMAP (0.12 g, 1.15 mmol, 1.00 eq), 1-Ethyl-3-(3-dimethylaminopropyl)carbodiimid (0.24 g, 1.27 mmol, 1.10 eq) and 4-tert-butyl-2-methoxy benzoic acid (64b, 0.24 g, 1.15 mmol, 1.0 eq) were added. The mixture was stirred for 16 h at room temperature. Then 25 mL 5% aqueous hydrochloric acid was added and the aqueous layer was extracted three times with 15 mL EtOAc. The combined organic layers were dried over Na₂SO₄ and the solvent was removed in vacuo. Further purification was performed by crystallization from hexane/ethyl acetate to obtain 69b as pale yellow solid (0.324 g, 81%). 1H NMR (250 MHz, DMSO-d₆): δ=8.62 (t, J=6.0 Hz, 1H), 7.73 (d, J=8.0 Hz, 1H), 7.27 (d, J=8.3 Hz, 1H), 7.07 (dd, J=10.1, 2.0 Hz, 3H), 6.97 (t, J=7.2 Hz, 1H), 4.47 (d, J=6.0 Hz, 2H), 3.94 (s, 3H), 1.31 (s, 9H). ¹³C NMR (75 MHz, DMSO-d₆) δ=164.89, 156.98, 155.90, 150.38, 150.31, 132.32, 130.41, 129.61, 119.72, 117.52, 109.08, 106.97, 55.89, 34.91, 30.90.

### 4-(tert-Butyl-3-methoxy)-N-(2-chloro-4-(methylsulfonamido)benzyl)benzamide (76a)

N-(4-Amino-2-chlorobenzyl)-4-(tert-butyl-2-methoxy)benzamide 69b (0.32 g, 0.93 mmol, 1.00 eq) was dissolved in 20 mL CHCl₃ and 5 mL pyridine were added. Mesyl chloride (70, 0.09 mL, 1.12 mmol, 1.20 eq) was carefully added. The mixture was stirred for two hours at room temperature. Then 15 mL 10% aqueous hydrochloric acid was added and extracted three times by 30 mL ethyl acetate. The combined organic layers were dried over Na₂SO₄ and the solvent was removed in vacuo. Further purification was performed by crystallization from hexane/ethyl acetate to obtain 76a as pale brown solid (0.172 g, 35%). ¹H NMR (250 MHz, DMSO-d₆) δ=9.97 (s, 1H), 8.72 (t, J=6.0 Hz, 1H), 7.79 (d, J=8.0 Hz, 1H), 7.36 (dd, J=14.9, 5.2 Hz, 2H), 7.25-7.13 (m, 3H), 4.56 (d, J=6.0 Hz, 2H), 4.00 (s, 3H), 3.08 (s, 3H), 1.37 (s, 9H). ¹³C NMR (126 MHz, DMSO-d₆) δ=165.47, 157.45, 156.36, 138.79, 132.71, 132.32, 130.85, 129.87, 120.27, 117.99, 109.54, 60.22, 56.36, 35.39, 31.37, 21.24, 14.56.

### 4-(tert-Butyl-2-hydroxy)-N-(2-chloro-4-(methylsulfonamido)benzyl)benzamide (77)

4-(tert-Butyl-2-methoxy)-N-(2-chloro-4-(methylsulfonamido)benzyl)benzamide (76a, 0.17 g, 0.40 mmol, 1.0 eq) was dissolved in 30 mL DCM and BBr₃ (4.05 mL, 4.05 mmol in DCM, 10.0 eq) was added at 0° C. The mixture was stirred for 16 hours at room temperature and then diluted in 30 mL iced water. The pH was adjusted to 6 with NaHCO₃ and the mixture was extracted three times with 30 mL ethyl acetate at a time. The combined organic layers were concentrated in vacuo. Further purification was performed by crystallization from hexane/ethyl acetate to obtain 77 as colorless solid (0.098 g, 60%). ¹H NMR (500 MHz, DMSO-d₆) δ=12.30 (s, 1H), 9.95 (s, 1H), 9.21 (t, J=5.5 Hz, 1H), 7.84 (d, J=8.4 Hz, 1H), 7.35-7.24 (m, 2H), 7.15 (d, J=8.5 Hz, 1H), 6.98-6.93 (m, 1H), 6.89 (s, 1H), 4.51 (d, J=5.6 Hz, 2H), 3.02 (s, 3H), 1.26 (s, 9H). ¹³C NMR (126 MHz, DMSO-d₆) δ=168.84, 159.78, 157.29, 138.57, 132.42, 131.05, 129.75, 127.69, 119.71, 118.24, 116.17, 113.95, 112.50, 34.65, 30.71, 30.49. HRMS (MALDI): m/z calculated 411.11398 for C₁₉H₂₄ClN₂O₄S, found 411.11373 [M+H]⁺.

### N-(4-Amino-2-chlorobenzyl)-4-(tert-butyl-3-methoxy)benzamide (69c)

4-Amino-2-chlorobenzamine (58k, 0.23 g, 0.15 mmol, 1.3 eq) was dissolved in 30 mL dry CHCl₃. 4-DMAP (0.12 g, 1.15 mmol, 1.00 eq), 1-Ethyl-3-(3-dimethylaminopropyl)carbodiimid (0.24 g, 1.27 mmol, 1.10 eq) and 4-tert-butyl-3-methoxy benzoic acid (64a, 0.24 g, 1.15 mmol, 1.0 eq) were added. The mixture was stirred for 16 h at room temperature. Then 25 mL 5% aqueous hydrochloric acid was added and the aqueous layer was extracted three times with 15 mL EtOAc at a time. The combined organic layers were dried over Na₂SO₄ and the solvent was removed in vacuo. Further purification was performed by crystallization from hexane/ethyl acetate to obtain 69c as pale yellow solid (0.312 g, 78%). ¹H NMR (250 MHz, DMSO-d₆): δ=8.84 (t, J=5.7 Hz, 1H), 7.45 (d, J=9.7 Hz, 2H), 7.29 (d, J=7.9 Hz, 1H), 7.13 (d, J=8.4 Hz, 1H), 6.86 (s, 1H), 6.72 (d, J=8.1 Hz, 1H), 4.42 (d, J=5.7 Hz, 2H), 3.86 (s, 3H), 1.34 (s, 9H). MS (ESI+): m/z 369.10 ([M+Na]⁺, 100)

### 4-(tert-Butyl-3-methoxy)-N-(2-chloro-4-(methylsulfonamido)benzylbenzamide (76b)

N-(4-Amino-2-chlorobenzyl)-4-(tert-Butyl-3-methoxy)benzamide 69c (0.31 g, 0.89 mmol, 1.00 eq) was dissolved in 20 mL CHCl₃ and 5 mL pyridine were added. Mesyl chloride 70 (0.07 mL, 1.07 mmol, 1.20 eq) was carefully added. The mixture was stirred for two hours at room temperature. Then 15 mL 10% aqueous hydrochloric acid was added and extracted three times by 30 mL ethyl acetate. The combined organic layers were dried over Na₂SO₄ and the solvent was removed in vacuo. Further purification was performed by crystallization from hexane/ethyl acetate to obtain 76b as pale brown solid (0.315 g, 83%). ¹H NMR (250 MHz, DMSO-d₆) δ=9.92 (s, 1H), 8.95 (t, J=5.8 Hz, 1H), 7.46 (dd, J=9.6, 1.6 Hz, 2H), 7.34-7.26 (m, 3H), 7.15 (dd, J=8.4, 2.2 Hz, 1H), 4.49 (d, J=5.7 Hz, 2H), 3.87 (s, 3H), 3.01 (s, 3H), 1.34 (s, 9H). ¹³C NMR (126 MHz, DMSO-d₆) δ=172.05, 170.40, 166.16, 158.02, 140.79, 138.38, 133.14, 132.33, 131.72, 129.59, 126.16, 119.82, 119.39, 118.34, 110.73, 59.79, 55.42, 34.68, 29.43.

### 4-(tert-Butyl-3-hydroxy)-N-(2-chloro-4-(methylsulfonamido)benzyl)benzamide (78)

4-(tert-Butyl-3-methoxy)-N-(2-chloro-4-(methylsulfonamido)benzyl)benzamide (76b, 0.31 g, 0.74 mmol, 1.0 eq) was dissolved in 50 mL DCM and BBr₃ (7.40 mL, 7.40 mmol in DCM, 10.0 eq) was added at 0° C. The mixture was stirred for 16 hours at room temperature and then diluted in 50 mL iced water. The pH was adjusted to 6 with NaHCO₃ and the mixture was extracted three times with 30 mL ethyl acetate at a time. The combined organic layers were concentrated in vacuo. Further purification was performed by crystallization from hexane/ethyl acetate to obtain 78 as colorless solid (0.145 g, 48%). ¹H NMR (500 MHz, DMSO-d₆) δ=9.92 (s, 1H), 9.62 (s, 1H), 8.81 (t, J=5.8 Hz, 1H), 7.31-7.27 (m, 2H), 7.27-7.25 (m, 2H), 7.21 (d, J=8.1 Hz, 1H), 7.15 (dd, J=8.4, 2.2 Hz, 1H), 4.45 (d, J=5.8 Hz, 2H), 3.01 (s, 3H), 1.35 (s, 9H). ¹³C NMR (126 MHz, DMSO-d₆) δ=170.82, 166.91, 156.27, 139.24, 138.78, 133.49, 132.72, 132.23, 129.83, 126.61, 120.22, 118.72, 117.77, 115.88, 34.93, 29.61. HRMS (MALDI): m/z calculated 411.11398 for C₁₉H₂₄ClN₂O₄S, found 411.11383 [M+H]⁺.

### N-(4-Amino-2-(trifluoromethyl)benzyl)-4-(tert-butyl)benzamide (69d)

1-(4-Amino-2-(trifluoromethyl)phenyl)methanamine 58z (0.25 g, 1.3 mmol, 1.1 eq) was dissolved in 7 mL CHCl₃, 7 mL NEt₃ were added and the mixture was cooled to 0° C. 4-tert-Butylbenzoyl chloride 630 (0.28 mL, 1.4 mmol, 1.1 eq) was slowly added over 10 min and the mixture was stirred for two hours at room temperature. Then, 10 mL 10% aqueous hydrochloric acid were added, phases were separated and the aqueous layer was washed with 10 mL EtOAc. The aqueous layer was brought to pH 10 with Na₂CO₃ solution and extracted three times with 10 mL EtOAc at a time. The combined organic layers were dried over Na₂SO₄ and the solvent was evaporated in vacuum. Further purification was performed by column chromatography with hexane/EtOAc (90:10) as mobile phase to obtain 69d as yellow solid (0.35 g, 84%). R_{f}(hexane/EtOAc=90:10)=0.27. ¹H NMR (250 MHz, DMSO-d₆) δ=8.77 (t, J=5.7 Hz, 1H), 7.87-7.79 (m, 2H), 7.48 (d, J=8.5 Hz, 2H), 7.13 (d, J=8.4 Hz, 1H), 6.90 (d, J=2.3 Hz, 1H), 6.74 (dd, J=8.3, 2.1 Hz, 1H), 5.44 (s, 2H), 4.47 (d, J=5.3 Hz, 2H), 1.30 (s, 9H). ¹³C NMR (75 MHz, DMSO-d₆) δ=166.14, 154.04, 147.81, 131.50, 129.71, 127.15, 125.05, 123.08, 116.80, 110.55, 110.47, 34.60, 30.94.

### 4-(tert-Butyl)-N-(2-trifluoromethyl-4-(methylsulfonamido)benzyl)benzamide(80)

N-(4-Amino-2-(trifluoromethyl)benzyl)-4-(tert-butyl)benzamide 69d (0.35 g, 1.0 mmol, 1.00 eq) was dissolved in 50 mL THF and 5 mL pyridine were added. Mesyl chloride (70, 0.23 mL, 3.0 mmol, 3.0 eq) was carefully added. The mixture was stirred overnight at room temperature. Then 50 mL 10% aqueous hydrochloric acid was added and extracted three times by 20 mL ethyl acetate. The combined organic layers were dried over Na₂SO₄ and the solvent was removed in vacuo. Further purification was performed by column chromatography with hexane/EtOAc (67:33) as mobile phase to obtain 80 as pale pink solid (0.009 g, 22%). R_{f}(hexane/EtOAc=67:33)=0.19. ¹H NMR (500 MHz, DMSO-d₆) δ=10.06 (s, 1H), 9.00 (t, J=5.8 Hz, 1H), 7.89-7.82 (m, 2H), 7.54-7.43 (m, 5H), 4.59 (d, J=5.5 Hz, 2H), 3.03 (s, 3H), 1.30 (s, 9H). ¹³C NMR (126 MHz, DMSO-d₆) δ=166.42, 154.33, 137.52, 132.71, 131.25, 129.78, 127.24, 125.20, 123.13, 116.72, 116.67, 34.69, 30.98. HRMS (MALDI): m/z calculated 429.14542 for C₂₀H₂₄F₃N₂O₃S, found 429.14523 [M+H]⁺.

### 4-(tert-Buty)-N-(2-chloro-4-(isopropylsulfonamide)benzyl)-benzamide(81)

N-(4-Amino-2-chlorobenzyl)-4-(tert-butyl)benzamide 69a (0.154 g, 0.49 mmol, 1.0 eq) was dissolved in 20 mL THF and 2 mL pyridine was added. Isopropylsulfonyl chloride 86a (0.57 mL, 4.89 mmol, to eq.) was carefully added, and the mixture was stirred for 24 h at room temperature. Then 15 mL of 10% aqueous hydrochloric acid was added and the mixture was extracted three times with 30 mL of EtOAc at a time. The combined organic layers were dried over Na₂SO₄ and the solvent was evaporated in vacuo. Further purification was performed by column chromatography using hexane/EtOAc (67:33) as mobile phase to obtain 81 yellow solid (0.041 g, 20%). R_{f}(hexane/EtOAc=67:33)=0.20. ¹H NMR (500 MHz, DMSO-d₆) δ=9.95 (s, 1H), 8.92 (t, J=5.7 Hz, 1H), 7.84 (d, J=8.4 Hz, 2H), 7.49 (d, J=8.4 Hz, 2H), 7.36-7.24 (m, 2H), 7.17 (dd, J=8.5, 2.1 Hz, 1H), 4.46 (d, J=5.7 Hz, 2H), 1.30 (s, 9H). ¹³C NMR (126 MHz, DMSO-d₆) δ=166.28, 154.21, 138.65, 132.33, 131.37, 131.34, 129.56, 127.20, 125.13, 119.19, 117.78, 51.59, 34.65, 30.96, 16.10. HRMS (MALDI): m/z calculated 423.15037 for CH₂₁H₂₈ClN₂O₃S, found 423.14967 [M+H]⁺.

### 4-(tert-Butyl)-N-(2-chloro-4-(ethylsulfonamido)benzyl)-benzamide(82)

N-(4-Amino-2-chlorobenzyl)-4-(tert-butyl)benzamide 69a (0.16 g, 0.51 mmol, 1.0 eq) was dissolved in 20 mL THF and 2 mL pyridine was added. Ethanesulfonyl chloride 86b (0.5 mL, 5.1 mmol, 10 eq) was carefully added, and the mixture was stirred for 24 h at room temperature. Then 15 mL of 10% aqueous hydrochloric acid was added and the mixture was extracted three times with 30 mL of EtOAc at a time. The combined organic layers were dried over Na₂SO₄ and the solvent was evaporated in vacuo. Further purification was performed by column chromatography using hexane/EtOAc (50:50) as mobile phase to obtain 82 as yellow solid (0.125 g, 60%). R_{f}(hexane/EtOAc=50:50)=0.51. H NMR (500 MHz, DMSO-d₆) δ=9.98 (s, 1H), 8.93 (t, J=5.8 Hz, 1H), 7.84 (d, J=8.4 Hz, 2H), 7.49 (d, J=8.4 Hz, 2H), 7.29 (d, J=8.4 Hz, 1H), 7.27 (d, J=2.1 Hz, 1H), 7.15 (dd, J=8.4, 2.1 Hz, 1H), 4.47 (d, J=5.7 Hz, 2H), 3.11 (q, J=7.3 Hz, 2H), 1.30 (s, 9H), 1.18 (t, J=3.6 Hz, 3H). ¹³C NMR (126 MHz, DMSO-d₆) δ=166.27, 154.20, 138.42, 132.34, 131.51, 131.34, 129.57, 127.19, 125.13, 119.35, 117.89, 59.78, 45.29, 39.52, 34.65, 30.96, 8.02. HRMS (MALDI): m/z calculated 409.13472 for C₂₀H₂₆ClN₂O₃S, found 409.13444 [M+H]⁺.

### N-(4-Amino-2-chlorobenzyl)-4-(dimethylamino)benzamide(69e)

1-(4-Amino-1-chlorophenyl)methanamine 58k (0.30 g, 1.92 mmol, 1.1 eq) was dissolved in 10 mL DMF. 10 mL NEt₃ was added and the mixture was cooled to 0° C. 4-Dimethylaminobenzoyl chloride 63p (0.32 mL, 1.73 mmol, 1.0 eq) was slowly added over 10 min and the mixture was stirred for 5 h at room temperature. Then 50 mL of 10% aqueous hydrochloric acid was added, phases were separated and the aqueous layer was brought to pH to with Na₂CO₃ solution and extracted three times with 80 mL of EtOAc at a time. The combined organic layers were dried over Na₂SO₄ and the solvent was evaporated in vaccuo. Further purification was performed by column chromatography with EtOAc/hexane/NEt₃ (65:33:2) to obtain 69e as a yellow solid (0.205 g, 39%). R_{f}(EtOAc/hexane/NEt₃=65:33:2)=0.47. ¹H NMR (250 MHz, DMSO-d₆) δ=8.42 (t, J=5.7 Hz, 1H), 7.76 (d, J=9.0 Hz, 2H), 6.98 (d, J=8.3 Hz, 1H), 6.70 (d, J=9.0 Hz, 2H), 6.61 (d, J=2.2 Hz, 1H), 6.47 (dd, J=8.3, 2.3 Hz, 1H), 5.24 (s, 2H), 4.34 (d, J=5.7 Hz, 2H). ¹³C NMR (75 MHz, DMSO-d₆) δ=166.25, 152.23, 148.96, 132.34, 129.62, 128.71, 123.13, 121.02, 113.65, 112.70, 110.88, 39.87.

### 4-(Dimethylamino)-N-(2-chloro-4-(methylsulfonamido)benzyl)benzamide(83)

N-(4-Amino-2-chlorobenzyl)-4-(dimethylamino)benzamide 69e (0.204 g, 0.67 mmol, 1.0 eq) was dissolved in 30 mL THF and 3 mL pyridine was added. Mesyl chloride 70 (0.27 mL, 3.36 mmol, 5.0 eq) was carefully added, and the mixture was stirred for 2 h at room temperature. Then 15 mL of 10% aqueous hydrochloric acid was added and the mixture was extracted three times with 30 mL EtOAc at a time. The combined organic layers were dried over Na₂SO₄ and the solvent was evaporated in vacuo. Further purification was performed by column chromatography using EtOAc as mobile phase. Further crystallization was performed in DCM/hexane and acetone/water to obtain 83 as a yellow solid (0.064 g, 25%). R_{f}(EtOAc)=0.64. ¹H NMR (500 MHz, MeOD-d₄) δ=7.76 (d, J=9.0 Hz, 2H), 7.33 (t, J=5.5 Hz, 2H), 7.14 (dd, J=8.4, 2.2 Hz, 1H), 6.73 (d, J=9.0 Hz, 2H), 5.49 (s, 1H), 4.59 (s, 2H), 3.02 (s, 6H), 2.96 (s, 3H). ¹³C NMR (126 MHz, MeOD-d₄) δ=170.50, 154.38, 139.71, 134.72, 133.51, 130.77, 129.89, 121.74, 121.54, 119.63, 112.13, 41.90, 40.22, 39.32. HRMS (MALDI): m/z calculated 380.08302 for C₁₇H₁₉ClN₃O₃S, found 380.08274 [M−H]⁻.

### 4-((1-Trifluoromethyl)cycloprop-1-yl)benzoic acid(64i)

Pd(OAc)₂ (0.045 mmol, 0.01 g, 3 mol %), Xantphos (0.045 mmol, 0.01 g, 3 mol %) were dissolved in 10 mL DMF. Afterwards formic acid (10.6 mmol, 0.4 mL, 7.0 eq) and 1-Bromo-4-(1-(trifluoromethyl)cycloprop-1-yl)benzene 87 (1.5 mmol, 0.4 g, 1.0 eq) were added dropwise. Then 1-Ethyl-3-(3-dimethylaminopropyl)carbodiimide and Triethylamine were added to the mixture. The mixture was stirred at 50° C. for 20 h. After cooling the mixture down to room temperature 10 ml of 10% aqueous hydrochloric acid was added and the mixture was extracted three times with 30 mL of EtOAc at a time. The combined organic layers were extracted with 30 ml of a saturated aqueous solution of Na₂CO₃ at a time. Then the combined aqueous layers were brought to pH 1 with concentrated hydrochloric acid and extracted three times with 30 ml of EtOAc at a time. The combined organic layers were dried over Na₂SO₄ and the solvent was evaporated in vacuo to obtain 64i without further purifications as a beige solid (0.177 g, 51%). ¹H NMR (300 MHz, DMSO-d₆) δ=13.04 (s, 1H), 8.00-7.93 (m, 2H), 7.60 (d, J=8.1 Hz, 2H), 1.40 (dd, J=7.0, 5.0 Hz, 2H), 1.23-1.11 (m, 2H). ¹³C NMR (75 MHz, DMSO-d₆) δ=166.85, 140.01, 131.17, 130.85, 129.40, 128.15, 27.75-27.32, 9.78-9.75.

### N-(4-Amino-2-chlorobenzyl)-4-((1-trifluoromethyl)cycloprop-1-yl)benzamide(69f)

1-(4-Amino-1-chlorophenyl)methanamine 58k (0.31 g, 2.0 mmol, 3.0 eq), 1-Ethyl-3-(3-dimethylaminopropyl)carbodiimide (0.31 g, 2.0 mmol, 3.0 eq) and 4-(Dimethylamino)pyridine (0.66 mmol, 0.08 g, 1.0 eq) were dissolved in 10 mL CHCl₃ and 1 mL DMF. Then 4-((1-Trifluoromethyl)cycloprop-1-yl)benzoic acid 64i (0.15 g, 0.66 mmol, 1.0 eq) dissolved in 5 mL CHCl₃ and 0.5 mL DMF was slowly added over 10 min and the mixture was stirred for 5 h at 60° C. After cooling the mixture to room temperature 10 mL of 10% aqueous hydrochloric acid was added, phases were separated and the aqueous layer was brought to pH 10 with Na₂CO₃ solution and extracted three times with 20 mL of EtOAc at a time. The combined organic layers were dried over Na₂SO₄ and the solvent was evaporated in vaccuo. Further purification was performed by column chromatography with hexane/EtOAC (57:43) as a mobile phase to obtain 69f as a yellow solid (0.123 g, 50%). R_{f}(hexane/EtOAc=57:43)=0.45. ¹H NMR (300 MHz, acetone-d₆) δ=7.95-7.90 (m, 2H), 7.57 (d, J=8.1 Hz, 2H), 7.15 (d, J=8.3 Hz, 1H), 6.72 (d, J=2.3 Hz, 1H), 6.58 (dd, J=8.3, 2.3 Hz, 1H), 4.85 (s, 1H), 4.54 (d, J=5.6 Hz, 2H), 1.39 (dd, J=6.9, 5.0 Hz, 2H), 1.18-1.12 (m, 2H). ¹³C NMR (75 MHz, MeOD-d₄) δ=169.60, 149.99, 140.91, 135.73, 134.95, 132.42, 131.42, 128.50, 124.97, 116.42, 114.75, 42.29, 29.28, 28.83, 10.41

### 4-((1-Trifluoromethyl)cycloprop-1-yl)-N-(2-chloro-4-(methylsulfonamido)benzyl)benzamide(84)

N-(4-Amino-2-chlorobenzyl)-4-((1-trifluoromethyl)cycloprop-1-yl)-benzamide 69f (0.1 g, 0.028 mmol, 1.0 eq) was dissolved in 15 mL THF and 1.5 mL pyridine was added. Mesyl chloride 70 (0.22 mL, 2.8 mmol, 10.0 equiv.) was carefully added, and the mixture was stirred for 24 h at room temperature. Then 15 mL of 10% aqueous hydrochloric acid was added and the mixture was extracted three times with 30 mL EtOAc at a time. The combined organic layers were dried over Na₂SO₄ and the solvent was evaporated in vacuo. Further purification was performed by column chromatography using hexane/EtOAc (50:50) as mobile phase to obtain 84 as white solid (0.052 g, 41%). R_{f}(hexane/EtOAc=50:50)=0.36. ¹H NMR (500 MHz, DMSO-d₆) δ=9.93 (s, 1H), 9.04 (s, 1H), 7.91 (d, J=8.3 Hz, 2H), 7.57 (d, J=8.1 Hz, 2H), 7.32 (d, J=8.4 Hz, 1H), 7.26 (d, J=2.1 Hz, 1H), 7.15 (dd, J=8.4, 2.1 Hz, 1H), 4.48 (d, J=5.7 Hz, 2H), 3.02 (s, 3H), 1.38 (s, 2H), 1.17 (s, 2H). ¹³C NMR (126 MHz, MeOD-d₄) δ=169.74, 141.13, 139.98, 135.45, 134.93, 132.85, 132.52, 131.10, 128.53, 121.72, 119.59, 42.16, 39.37, 29.21-28.95, 10.44-10.43. HRMS (MALDI): m/z calculated 447.07515 for C₁₉H₁₉ClF₃N₂O₃S, found 447.07455 [M+H]⁺.

### N-(4-Amino-2-chlorobenzyl)-4-(pyrrolidine)-benzamide(69g)

1-(4-Amino-1-chlorophenyl)methanamine 58k (0.49 g, 3.1 mmol, 3.0 eq), 1-Ethyl-3-(3-dimethylaminopropyl)carbodiimide (0.49 g, 3.1 mmol, 3.0 eq) and 4-(Dimethylamino)pyridine (1.1 mmol, 0.13 g, 1.0 eq) were dissolved in 20 mL CHCl₃ and 2 mL DMF. Then 4-pyrrolidinebenzoic acid (0.2 g, 1.1 mmol, 1.0 eq) dissolved in 5 ml CHCl₃ and 0.5 mL DMF was slowly added over 10 min and the mixture was stirred for 5 h at 60° C. After cooling the mixture down to room temperature 10 mL of 10% aqueous hydrochloric acid was added, phases were separated and the aqueous layer was brought to pH 10 with Na₂CO₃ solution and extracted three times with 20 mL EtOAc at a time. The combined organic layers were dried over Na₂SO₄ and the solvent was evaporated in vaccuo. Further purification was performed by column chromatography with EtOAc/hexane (86:14) as mobile phase to obtain 69g as yellow solid (0.100 g, 29%). R_{f}(EtOAc/hexane=86:14)=0.45. ¹H NMR (250 MHz, DMSO-d₆) δ=8.39 (s, 1H), 7.76 (d, J=8.8 Hz, 2H), 6.98 (d, J=8.3 Hz, 1H), 6.61 (d, J=2.2 Hz, 1H), 6.53 (d, J=8.9 Hz, 2H), 6.47 (dd, J=8.3, 2.3 Hz, 1H), 5.24 (s, 2H), 4.35 (d, J=5.7 Hz, 2H), 3.27 (d, J=7.2 Hz, 4H), 1.96 (t, J=4.8 Hz, 4H). ¹³C NMR (75 MHz, DMSO-d₆) δ=162.32, 149.50, 148.89, 132.23, 129.53, 128.75, 123.11, 120.31, 113.53, 112.59, 110.54, 47.21, 35.78, 24.96.

### 4-(Pyrrolidine)-N-(2-chloro-4-(methylsulfonamido)benzyl)benzamide(85)

N-(4-Amino-2-chlorobenzyl)-4-(pyrrolidine)-benzamide 69g (0.1 g, 0.03 mmol, 1.0 eq) was dissolved in 15 mL THF and 2 mL pyridine was added. Mesyl chloride 70 (0.12 mL, 1.55 mmol, 5.0 eq) was carefully added, and the mixture was stirred for 2 h at room temperature. Then 15 mL of 10% aqueous hydrochloric acid was added and the mixture was extracted three times with 30 mL of EtOAc at a time. The combined organic layers were dried over Na₂SO₄ and the solvent was evaporated in vacuo. Further purification was performed by column chromatography using EtOAc/hexane (67:33) yielding 85 a white solid (0.033 g, 26%). R_{f}(EtOAc/hexane=67:33)=0.37. ¹H NMR (500 MHz, DMSO-d₆) δ=9.91 (s, 1H), 8.60 (s, 1H), 7.77 (d, J=8.8 Hz, 2H), 7.28 (d, J=8.4 Hz, 1H), 7.25 (d, J=2.1 Hz, 1H), 7.14 (dd, J=8.4, 2.2 Hz, 1H), 6.54 (d, J=8.9 Hz, 2H), 4.44 (d, J=5.5 Hz, 2H), 3.28 (s, 4H), 3.01 (s, 3H), 1.98-1.94 (m, 4H). ¹³C NMR (126 MHz, DMSO-d₆) δ=166.38, 149.61, 138.20, 132.30, 132.19, 129.43, 128.81, 119.97, 119.76, 118.29, 110.59, 47.23, 42.98, 36.56, 24.98. HRMS (MALDI): m/z calculated 408.11432 for C₁₉H₂₃ClN₃O₃S, found 408.11361 [M+H]⁺.

### Example 4: Virtual Inspection

Binding of compound 57 was analyzed in silico by molecular docking using the X-ray structures of sEH and FXR containing the ligands from which lead compound 5 was constructed (compound 3/PDB-ID: 4QE8 for FXR and compound 2/PDB-ID: 3128 for sEH). The resulting binding modes (shown in FIG. 2) are in congruence with the SAR of N-benzylbenzamides 5-57 on both targets. In the binding mode of 57 in FXR (FIG. 2A), the tert-butyl moiety tightly fits into the binding pocket and mediates receptor activation through stabilization of helix 12. The adjacent phenyl ring is well positioned into the lipophilic pocket that does not allow variations in 2- or 3-position. The sulfonamide occupies a hydrophilic region and does not exhibit specific interactions, which explains the wide tolerability of hydrophilic moieties in this position of the benzyl moiety. The amide moiety forms no directed H-bonds which is also the case for the reference ligand 3 in the FXR X-ray structure 4QE8. The methylene bridge is bound in proximity to Leu287 explaining the enhanced potency of compound 32 which carries an additional methyl group in this position. The chlorine atom points towards a tight subpocket defined by Ile352 and the phenolic moiety of Tyr369 (supporting figure Si) that tolerates chlorine or fluorine (30) but no purely lipophilic residues as the methyl substituent in 29.

The proposed binding mode of 57 to sEH (FIG. 2B) reveals that its amide group interacts with the catalytic residues Tyr383, Tyr466, and Asp335. The methylene bridge is located in a narrow tunnel which does not allow any structural modifications. The chlorine substituent of the benzyl moiety points towards a lipophilic pocket and is crucial for binding. Similar to the FXR binding mode, the sulfonamide moiety binds in a more hydrophilic subpocket does not form specific interactions. The 4-tert-butyl phenyl residue is located in a tight hydrophobic pocket offering space for substituents in position 4 or 3, but not in 2-position of the aromatic ring.

To study the selectivity profile of 57 amongst related nuclear receptors, the inventors determined its activity on PPARs, LXRs, RXRs, RARs, PXR and VDR at to 10 μM concentration in Gal4-hybrid reporter gene assays for the respective receptors (FIG. 3A). 57 was inactive on PPARα and PPARδ, both LXR subtypes as well as RXRα. Only on PPARγ 57 exhibited weak partial agonism with an EC₅₀ value of 14.7±0.9 μM and, therefore, is highly selective for FXR amongst nuclear receptors (selectivity ≥720). Moreover, 57 displayed no cytotoxic activity up to a concentration of 100 μM in a water soluble tetrazolium (WST-1) assay (FIG. 3B). To estimate metabolic stability, 57 was incubated with liver microsomes of Wistar rats which revealed an acceptable stability with >50% of the compound remaining after 60 minutes (FIG. 3C). Still, the inventors studied metabolic conversion of 57 more in detail in vitro and identified its metabolites (FIG. 6, scheme 10). According to LC-MS-MS analysis (supporting FIGS. 82-86), 57 is metabolized by hydrolysis of the sulfonamide moiety resulting in aniline 69a, by hydroxylation on the tert-butylbenzamide moiety which can lead to the three isomers 77, 78 and 79 (FIG. 6, scheme 10) and by hydroxylation on the aromatic ring of the benzylsubstituent. The inventors synthesized 77 and 78 carrying a hydroxyl group on the benzamide aromatic ring but both isomers were not detectable in the metabolized residue confirming 79 as metabolite of 57. Metabolite 69a retains considerable potency activating FXR with an EC₅₀ value of 0.046±0.006 μM and inhibiting sEH with an IC₅₀ value of 0.040±0.006 μM. Hence, metabolite 69a may contribute to the pharmacodynamic activity of dual modulation in vivo and prolong the pharmacologic effect of the original compound 57.

To evaluate the FXR agonistic effect of 57 under less artificial conditions than in a reporter gene assay, the inventors also quantified the effect of the compound on FXR target gene expression in HepG2 hepatoma cells (FIG. 4A). For this purpose, the cells were incubated with the endogenous FXR agonist CDCA (b) at 50 μM, the partial agonist 57 at 0.1 μM and 1 μM or with DMSO (0.1%) as control for 8 or 16 hours and then FXR target gene mRNA was quantified. Data was analyzed according to the 2^{ΔΔCt} method and all results were normalized to the values of the house-keeping gene glycerinaldehyde 3-phosphate dehydrogenase (GAPDH). Gene expression of vehicle treated control cells was defined as 100%. 57 revealed a partial FXR agonistic profile and modulated all nine studied genes similar to the endogenous agonist CDCA (1b) but with smaller amplitude. Expression of the bile salt export protein (BSEP) was only moderately increased by 57 while induction of small hetero-dimer partner (SHP), peroxisome proliferator-activated receptor α (PPARα), fibroblast growth factor 19 (FGF 19), pyruvate dehydrogenase kinase 4 (PDK4) and liver-type fatty acid binding protein (fatty acid binding protein 1, FABP1) reached amplitudes of 50-80% of CDCA (1b). Moreover, 57 repressed cholesterol 7α-hydroxylase (CYP7A1), sterol regulatory element-binding protein 1c (SREBP1c) and fatty acid synthase (FAS) which are indirectly regulated by FXR via induction of SHP. Concerning CYP7A1, 57 behaved as partial agonist and caused less repression than 1b while repression of SREBP1c and FAS by 57 and 1b reached equal amplitudes. For all the genes, 57 caused an equal effect at 0.1 μM and 1 μM confirming saturation at these concentrations. In contrast, PPARγ target genes scavenger receptor 3B (CD36) and cytokine-like protein 2-19 (FAM3A) were not markedly modulated by 57 even at a higher concentration of 10 μM further confirming the compound's selectivity (FIG. 4B).

Inhibition of soluble epoxide hydrolase was also studied in a less artificial context in HepG2 cell homogenates by quantifying conversion of the deuterated sEH substrate 14.15-EET-d11 in presence of varying concentrations of inhibitor 57 (FIG. 4C). The compound exerted robust inhibition of cellular sEH with a statistically significant increase in the EET/DHET ratio even at 1 nM concentration. In the cellular context, 57 revealed an IC₅₀ value for sEH inhibition of approximately 10 nM.

### Example 5: In-Vivo Characterization

Encouraged by the high potency and very favorable in vitro characteristics of 57, the inventors conducted a pilot in vivo study of the compound in male wild-type C57BL6/J mice (FIG. 5). To record a pharmacokinetic profile and to evaluate pharmacodynamic data concerning FXR activation and sEH inhibition in vivo, six mice received a single dose of 57 (p.o., 10 mg/kg body weight). Three additional animals served as vehicle control.

The dual modulator 57 displayed favorable oral bioavailability (Cₘₐₓ=1182 ng/mL) and rapid uptake (tₘₐₓ=0.5 h) accompanied by a moderate half-life (t_{1/2}=0.7 h). Altogether, the single dose of 57 produced effective concentrations above the EC₅₀ (FXR) and IC₅₀ (sEH) values over approximately three to four hours (FIG. 5A). To evaluate pharmacodynamic effects of 57, mouse plasma was analyzed for EET/DHET ratio, and FXR target gene expression was determined in mouse livers 8 hours post application (FIG. 5B). The EET/DHET ratios for the 8.9- and 11.12-isomers were increased by approximately a factor 2 upon treatment with 57 indicating that sEH activity was inhibited by the dual modulator in vivo. Moreover, FXR target gene expression was altered in livers of mice receiving 57 with increased expression of BSEP (approx. 3-fold), SHP (approx. 4-fold) and FGF15 (approx. 2.5-fold) and reduced SREBP1c (approx. 5-fold) levels which also suggested FXR activation in vivo (FIG. 5C). CYP7A1 mRNA levels showed a slight trend to repression. Expression of the PPARγ target gene fatty acid transport protein (FATP) was not affected by 57 in vivo. Hence, the pilot animal study revealed acceptable pharmacokinetics and dearly indicated dual target engagement of 57 in vivo.

The growing incidence of NASH with hepatocellular carcinoma and liver cirrhosis as their most serious potential consequences represents a rapidly growing global health concern. While liver transplantation is the only effective therapy available to date, research on pharmacological options is very intensive. The FXR agonist OCA (1a) is leading the pipeline and holds much promise to be the first effective drug to counter NASH. It exhibited anti-steatotic and anti-fibrotic effects accompanied by several metabolic improvements in clinical trials.^{6,12} The dual PPARα/δ agonist elafibranor has successfully completed phase II trials and might succeed OCA as therapeutic option.³⁸ Still, the multifactorial nature of NASH involving steatosis, fibrosis and—importantly—inflammation might require a broader therapeutic strategy that addresses all participating factors. In light of the impressive efficacy of FXR activation on steatosis^{6,12} and promising reports on hepatic anti-steatotic as well as anti-inflammatory effects of sEH inhibition^{23,39}, the inventors assumed that combination of these strategies might be synergistic. Therefore, the inventors have developed a highly potent dual modulator of FXR and sEH with well-balanced activity.

The inventors successfully merged known pharmacophores of partial FXR agonists and sEH inhibitors to generate the lead structure 5 that displayed weak but statistically significant activity on both targets. The low fragment-like properties and structural flexibility of this lead compound allowed considerable structural variation to achieve optimization and, therefore, the moderate activity seemed sufficient. In four consecutive steps, the inventors systematically investigated the SAR of the compound class on FXR and sEH and reached strong optimization of dual potency. However, although the inventors identified several highly potent modulators of the single targets, no compound with low nanomolar potency on both targets was discovered in the systematic SAR study. For the final decisive optimization step we, therefore, combined structural elements of the most active agents on the single targets which led to the development of 57 as highly potent dual modulator with an EC₅₀ value of 20.4±4.2 nM for partial FXR activation and an IC₅₀ value of 4.1±0.4 nM for sEH inhibition. Broader in vitro characterization of 57 revealed a very favorable selectivity profile over related nuclear receptors and no cytotoxic activity up to a concentration of 100 μM. In evaluation of in vitro metabolism 57 turned out moderately stable which was confirmed by a moderate half-life in vivo. However, closer evaluation and characterization of the main metabolites indicates that aniline 69a formed by hydrolysis of the sulfonamide moiety of 57 possesses almost equal potency and is likely to be pharmacologically active prolonging the dual modulatory effect of 57.

In HepG2 cells, 57 exerted a partial induction of FXR target genes compared to the endogenous agonist CDCA (1b). This observation in non-transfected liver cells and the fact that the partial agonistic modulation was equal at 1 μM and 0.1 μM concentration confirmed the true partial FXR agonistic nature of the dual modulator 57. The clinical development of obeticholic acid (1a)⁶ has reported disturbed cholesterol homeostasis upon treatment with the full FXR agonist which seems due to strong repression of the FXR target gene cholesterol 7α hydroxylase (CYP7A1). This enzyme constitutes the first and rate-limiting enzyme in metabolic conversion of cholesterol to bile acids and, therefore, full FXR activation blocks one of the main pathways of cholesterol elimination. To avoid this side effect, partial FXR activation seems preferable.

The FXR target gene expression profile of HepG2 cells after stimulation with 57 suggests beneficial effects on NAFLD and NASH. Recent studies reported reduced serum levels of fibroblast factor 19 (FGF19) in NAFLD and NASH patients⁴⁰ and treatment with FGF9 improved insulin sensitivity, lowered body weight and decreased hepatic fat content in mice. Increased levels of FGF19 were observed under treatment with OCA (1a) and considered as important beneficial pharmacodynamic effect.¹² Induction of PPARα which is considered as major regulator of hepatic fatty acid degradation by β-oxidation⁴¹ combined with a repression of hepatic FAS causing reduced fatty acid de-novo synthesis is favorable for reducing hepatic steatosis. Notably, hepatic fat content in NAFLD and NASH is dominated by free fatty acids.⁴ This effect may be further enhanced by induction of PDK4 leading to reduced glycolysis and, consequently, to fatty acid utilization for energy generation.⁴² Liver-type fatty acid binding protein (FABP1) is involved in numerous physiological processes and globally affects lipid homeostasis. In liver, FABP1 has a cytoprotective role and counters oxidative cell damage.⁴³ Since oxidative stress in hepatocytes is a major factor in NAFLD/NASH development and manifestation, enhanced expression of FABP1 as exerted by 57 seems favorable in NASH.

Inhibitory potency of 57 on soluble epoxide hydrolase was also studied in a less artificial setting by quantifying the conversion of the deuterated sEH substrate 14.15-EET-d11 in HepG2 cell lysates. In this system, 57 possessed an IC₅₀ value of 1.6±0.5 nM which is in perfect agreement with the results obtained in the cell-free fluorescence-based assay on recombinant protein. Hence, the dual modulator 57 is equally potent in inhibiting the human sEH in presence of other proteins and cellular components from liver cells.

Encouraged by the promising in vitro profile the inventors applied 57 to a pilot in vivo study in male wild-type C57BL6/J mice to evaluate pharmacokinetics and pharmacodynamic effects of the dual modulator. 57 displayed favorably rapid uptake and oral bioavailability and although the molecule possessed a rather short half-life, the inventors observed active concentrations over a period of around 3.5-4 hours after a single oral dose of to mg/kg body weight. The quantification of FXR target gene mRNA in mouse livers 8 hours after application of 57 revealed a clear trend to upregulation of SHP and BSEP that only curtly failed to reach statistical significance and marked effects on the expression of FGF15 and SREBP1c. CYP7A1 displayed a slight trend to down-regulation. Particularly the induction of BSEP points to activation of FXR by 57 in vivo since this gene is almost exclusively regulated by FXR.⁴⁴⁻⁴⁶ Moreover, as discussed above, repression of SREBP1c and induction of FGF15 suggest favorable effects in NAFLD/NASH treatment Concerning sEH inhibition in vivo, the inventors evaluated the effect of 57 on the ratios of sEH substrates (EETs) to sEH products (DHETs) in plasma which were significantly shifted to EETs in mice receiving the dual modulator. This accumulation of EETs indicates that 57 inhibited sEH in vivo as well and might exhibit anti-inflammatory activity which would highly contribute to beneficial effects in NASH.

The here reported dual modulator 57 that partially activates FXR and inhibits sEH with low nanomolar potency is the first compound with such activity. Its pharmacodynamic effects with modulation of FXR target gene expression and EET/DHET ratios indicates that 57 hit both targets in vivo. For this unique activity, the dual modulator perfectly qualifies for larger animal models to study its therapeutic efficacy and the concept of dual FXR/sEH modulation in NASH and related metabolic or cardiovascular disorders.

Materials and Methods

Chemistry

General. All chemicals and solvents were of reagent grade and used without further purification unless otherwise specified. All reactions were conducted in oven-dried glassware under argon-atmosphere and in absolute solvents. NMR spectra were recorded on a Bruker AV 400, Bruker AV 300, Bruker am250xp or a Bruker AV 500 spectrometer (Bruker Corporation, Billerica, Mass., USA). Chemical shifts (δ) are reported in ppm relative to tetramethylsilane (TMS) as reference; multiplicity: s, singlet; d, doublet; dd, doublet of doublets; t, triplet; dt, doublet of triplets; m, multiplet; approximate coupling constants (J) are shown in hertz (Hz). Mass spectra were obtained on a VG Platform H (Thermo Fischer Scientific, Inc., Waltham, Mass., USA) using electrospray ionization (ESI). High resolution mass spectra were recorded on a MALDI LTQ ORBITRAP XL instrument (Thermo Fisher Scientific). Compound purity was analyzed on a Varian ProStar HPLC (SpectraLab Scientific Inc., Markham, ON, Canada) equipped with a MultoHigh₁₀₀ Phenyl-5μ 240+4 mm column (CS-Chromatographie Service GmbH, Langerwehe, Germany) using a gradient (H₂O/MeOH 80:20+0.1% formic acid isocratic for 5 min to MeOH+0.1% formic acid after additional 45 min and MeOH+0.1% formic acid for additional 10 min) at a flow rate of 1 mL/min and UV-detection at 245 nm and 280 nm. Compound 16 was analyzed by elemental analysis because no molecular ion was found in MS. All final compounds for biological evaluation had a purity ≥95%.

Preparation of Dual Modulator 57

### 1-(4-Amino-1-chlorophenyl)methanamine (58k)

LiAlH₄ (1 M in THF, 16.4 mL, 16.4 mmol, 2.5 eq) was cooled to 0° C. 4-Amino-2-chlorobenzonitrile 62 (1.0 g, 6.6 mmol, 1.0 eq) in 3 mL THF was slowly added to the mixture. After evolution of H₂ had ceased, the mixture was allowed to warm to room temperature and then refluxed for 16 h. After cooling to room temperature, the mixture was diluted with 10 mL THF and then cooled to 0° C. 1 mL 10% NaOH solution and 1.8 mL water were added dropwise. The colorless precipitate was filtered through celite and washed with 15 mL diethyl ether. Evaporation of the organic solvents from the filtrate yielded 58k as yellow oil (0.77 g, 75%). ¹H NMR (500 MHz, DMSO-d₆) δ=7.11 (d, J=8.2 Hz, 1H), 6.58 (d, J=2.2 Hz, 1H), 6.48 (dd, J=8.2, 2.2 Hz, 1H), 5.19 (s, 2H), 3.59 (s, 2H). ¹³C NMR (126 MHz, DMSO-d₆) δ=148.56, 132.33, 129.67, 127.74, 113.63, 112.72, 42.88.

### N-(4-Amino-1-chlorobenzyl)-4-(tert-butyl)benzamide (69a)

1-(4-Amino-1-chlorophenyl)methanamine 58k (0.31 g, 2.0 mmol, 1.1 eq) was dissolved in 10 mL CHCl₃, 5 mL NEt₃ were added and the mixture was cooled to 0° C. 4-tert-Butylbenzoyl chloride 630 (0.35 mL, 1.8 mmol, 1.0 eq) was slowly added over 10 min and the mixture was stirred for two hours at room temperature. Then, 50 mL to % aqueous hydrochloric acid were added, phases were separated and the aqueous layer was washed with 30 mL EtOAc. The aqueous layer was brought to pH to with Na₂CO₃ solution and extracted three times with 80 mL EtOAc at a time. The combined organic layers were dried over Na₂SO₄ and the solvent was evaporated in vacuum. Further purification was performed by column chromatography with petrolether/EtOAc (9:1) as mobile phase to obtain 69a as yellow solid (0.57 g, 97%). R_{f}(petrolether/EtOAc=2:1)=0.26. ¹H NMR (500 MHz, MeOH-d₄) δ=7.81 (dt, J=8.6, 2.3 Hz, 2H), 7.53 (dt, J=8.6, 2.3 Hz, 2H), 7.45 (d, J=8.3 Hz, 1H), 7.31 (d, J=2.2 Hz, 1H), 7.15 (dd, J=8.3, 2.2 Hz, 1H), 4.64 (s, 2H), 1.36 (s, 9H). ¹³C NMR (126 MHz, MeOH-d₄) δ=170.32, 156.67, 140.89, 135.23, 132.33, 131.39, 128.29, 126.59, 122.96, 120.82, 119.16, 42.13, 35.36, 31.54. HRMS (MALDI): m/z calculated 317.14152 for C₁₈H₂₁ ClN₂O, found 317.14130 [M+H]⁺.

### 4-(tert-Butyl)-N-(2-chloro-4-(methylsulfonamido)benzyl)benzamide (57)

N-(4-Amino-2-chlorobenzyl)-4-(tert-butyl)benzamide 69a (0.04 g, 0.12 mmol, 1.0 eq) was dissolved in 5 mL CHCl₃ and 0.5 mL pyridine were added. Mesyl chloride 70 (0.02 mL, 0.14 mmol, 1.2 eq) was carefully added and the mixture was stirred for two hours at room temperature. Then, 15 mL 10% aqueous hydrochloric acid were added and the mixture was extracted three times with 30 mL EtOAc at a time. The combined organic layers were dried over Na₂SO₄ and the solvent was evaporated in vacuum. Further purification was performed by column chromatography using petrolether/EtOAc (4:1) as mobile phase to obtain 57 as colorless solid (0.047 g, 66%). R_{f}(petrolether/EtOAc=2:1)=0.13. ¹H NMR (500 MHz, MeOH-d₄) δ=7.80 (dt, J=8.6, 2.0 Hz, 2H), 7.52 (dt, J=8.6, 2.0 Hz, 2H), 7.36 (d, J=8.4 Hz, 1H), 7.33 (d, J=2.2 Hz, 1H), 7.16 (dd, J=8.4, 2.3 Hz, 1H), 4.62 (s, 2H), 2.97 (s, 3H), 1.35 (s, 9H). ¹³C NMR (126 MHz, MeOH-d₄) δ=170.30, 156.55, 139.90, 134.88, 133.07, 132.46, 130.99, 128.29, 126.56, 121.75, 119.62, 42.06, 39.36, 35.81, 31.55. MS (ESI−): m/z 393.1 (100, [M−H]⁻). HRMS (MALDI): m/z calculated 395.11907 for C₁₉H₂₄ClN₂O₃S, found 395.11892 [M+H]⁺.

For preparation and characterization of compounds 4-56 and 77-78 as well as the respective intermediates please refer to supporting information.

Biological Evaluation

Full Length FXR Transactivation Assay

Plasmids: pcDNA3-hFXR contains the sequence of human FXR and was already published elsewhere.⁴⁷ pGL3 basic (Promega Corporation, Fitchburg, Wis., USA) was used as a reporter plasmid, with a shortened construct of the promotor of the bile salt export protein (BSEP) cloned into the SacI/NheI cleavage site in front of the luciferase gene.⁴⁸ pRL-V40 (Promega) was transfected as a control for normalization of transfection efficiency and cell growth. pSG5-hRXR was already published elsewhere as well.⁴⁹

Assay procedure: HeLa cells were grown in DMEM high glucose supplemented with 10% FCS, sodium pyruvate (1 mM), penicillin (100 U/mL) and streptomycin (100 μg/mL) at 37° C. and 5% CO₂. 24 h before transfection, HeLa cells were seeded in 96-well plates with a density of 8000 cells per well. 3.5 h before transfection, medium was changed to DMEM high glucose, supplemented with sodium pyruvate (1 mM), penicillin (100 U/mL), streptomycin (100 μg/mL) and 0.5% charcoal-stripped FCS. Transient transfection of HeLa cells with BSEP-pGL3, pRL-SV40 and the expression plasmids pcDNA3-hFXR and pSG5-hRXR was carried out using calcium phosphate transfection method. 16 h after transfection, medium was changed to DMEM high glucose, supplemented with sodium pyruvate (1 mM), penicillin (100 U/mL), streptomycin (100 μg/mL) and 0.5% charcoal-stripped FCS. 24 h after transfection, medium was changed to DMEM without phenol red, supplemented with sodium pyruvate (i mM), penicillin (100 U/mL), streptomycin (100 μg/mL), L-glutamine (2 mM) and 0.5% charcoal-stripped FCS, now additionally containing 0.1% DMSO and the respective test compound or 0.1% DMSO alone as untreated control. Each concentration was tested in triplicate wells and each experiment was repeated independently at least three times. Following 24 h incubation with the test compounds, cells were assayed for luciferase activity using Dual-Glo™ Luciferase Assay System (Promega) according to the manufacturer's protocol. Luminescence was measured with a Tecan Infinite M200 luminometer (Tecan Deutschland GmbH, Crailsheim, Germany). Normalization of transfection efficiency and cell growth was done by division of firefly luciferase data by renilla luciferase data multiplied by 1000 resulting in relative light units (RLU). Fold activation was obtained by dividing the mean RLU of the tested compound at a respective concentration by the mean RLU of untreated control. Relative activation was obtained by dividing the fold activation of the tested compound at a respective concentration by the fold activation of FXR full agonist GW4064 (1c) at 3 μM. EC₅₀ and standard error of the mean values were calculated with the mean relative activation values of at least three independent experiments by SigmaPlot 10.0 (Systat Software GmbH, Erkrath, Germany) using a four parameter logistic regression. The assay was validated with FXR agonists 1b (EC₅₀=18±1 μM, 88±3% rel. max. act.), is (EC₅₀=0.16±0.02 μM, 87±3% rel. max. act.) and 1c (EC₅₀=0.51±0.16 μM, 3 μM defined as 100%).¹⁵

sEH Activity Assay

The sEH inhibitory potency of the compounds were determined in a fluorescence-based 96-well sEH activity assay using recombinant human enzyme^{50,51}. Non-fluorescent PHOME (3-phenylcyano-(6-methoxy-2-naphthalenyl)methyl ester 2-oxiraneacetic acid; Cayman Chemicals) which can be hydrolyzed by the sEH to fluorescent 6-methoxynaphthaldehyde served as substrate. Recombinant human sEH (in Bis-Tris buffer, pH 7, with 0.1 mg/mL BSA containing a final concentration of 0.01% Triton-X 100) was pre-incubated with test compounds (in DMSO, final DMSO concentration: 1%) for 30 min at room temperature. Then, substrate was added (final concentration 50 μM) and hydrolysis of the substrate was determined by measuring fluorescent product formation on a Tecan Infinite F200 Pro (λₑₘ=330 nm, λₑₓ=465 nm) for 30 min (one point every minute). A blank control (no protein and no compound) as well as a positive control (no compound) were executed. All experiments were conducted in triplicate and repeated in at least three independent experiments. For IC₅₀ calculation, dose-response curves of increasing compound concentrations were recorded.

Hybrid reporter gene assays for PPARα/γ/δ, LXRα/β, RXRα/β/γ, RARα/β/γ, VDR and PXR Plasmids: The Gal4-fusion receptor plasmids pFA-CMV-hPPARα-LBD⁵², pFA-CMV-hPPARγ-LBD⁵², pFA-CMV-hPPARδ-LBD⁵², pFA-CMV-hLXRα-LBD³² and pFA-CMV-hLXRβ-LBD³² have been reported previously. The Gal4-fusion receptor plasmids pFA-CMV-hRXRα-LBD, pFA-CMV-hRXRβ-LBD, pFA-CMV-hRXRγ-LBD, pFA-CMV-hRARα-LBD, pFA-CMV-hRARβ-LBD, pFA-CMV-hRARγ-LBD, pFA-CMV-hVDR-LBD and pFA-CMV-hPXR-LBD coding for the hinge region and ligand binding domain (LBD) of the canonical isoform of the respective nuclear receptor (uniprot entries: hRXRα-P19793, residues 225-462; hRXRβ-P28702-1, residues 294-533; hRXRγ-P48443-1, residues 229-463; hRARα-P10276-1, residues 177-462; hRARβ-P10826-1, residues 177-455; hRARγ-P13631-1, residues 179-454; hVDR-P11473-1, residues 119-427; hPXR-O75469-1, residues 138-434) were constructed by integrating cDNA fragments obtained from PCR amplification of commercial cDNA (Source BioSience, Nottingham, UK) into the BamH1 cleavage site of the pFA-CMV vector (Stratagene, La Jolla, Calif., USA) and an afore inserted KpnI cleavage site. Frame and sequence of all fusion receptors were verified by sequencing. pFR-Luc (Stratagene) was used as reporter plasmid and pRL-SV40 (Promega) for normalization of transfection efficiency and cell growth.

Assay procedure: HEK293T cells were grown in DMEM high glucose, supplemented with 10% FCS, sodium pyruvate (1 mM), penicillin (100 U/mL) and streptomycin (100 μg/mL) at 37° C. and 5% CO₂. The day before transfection, HEK293T cells were seeded in 96-well plates (2.5·10⁴ cells/well). Before transfection, medium was changed to Opti-MEM without supplements. Transient transfection was carried out using Lipofectamine LTX reagent (Invitrogen) according to the manufacturer's protocol with pFR-Luc (Stratagene), pRL-SV40 (Promega) and pFA-CMV-hRXRα-LBD. 5 h after transfection, medium was changed to Opti-MEM supplemented with penicillin (100 U/mL), streptomycin (100 μg/mL), now additionally containing 0.1% DMSO and the respective test compound or 0.1% DMSO alone as untreated control. Each concentration was tested in triplicates and each experiment was repeated independently at least three times. Following overnight (12-14 h) incubation with the test compounds, cells were assayed for luciferase activity using Dual-Glo™ Luciferase Assay System (Promega) according to the manufacturer's protocol. Luminescence was measured with an Infinite M200 luminometer (Tecan Deutschland GmbH). Normalization of transfection efficiency and cell growth was done by division of firefly luciferase data by renilla luciferase data and multiplying the value by 1000 resulting in relative light units (RLU). Fold activation was obtained by dividing the mean RLU of a test compound at a respective concentration by the mean RLU of untreated control. Relative activation was obtained by dividing the fold activation of a test compound at a respective concentration by the fold activation of a respective reference agonist at 1 μM (PPARα: GW7647; PPARγ: pioglitazone; PPARδ: L165,041; LXRα/β: T0901317; RXRs: bexarotene; RARs: tretinoin; VDR calcitriol; PXR: SR12813). All hybrid assays were validated with the above mentioned reference agonists which yielded EC₅₀ values in agreement with literature.

FXR Target Gene Quantification (Quantitative Real-Time PCR)

FXR target gene quantification was performed as described previously.¹⁵ In brief, HepG2 cells were incubated with test compound 57 (0.1 μM and 1 μM) or 1b (50 μM) or 0.1% DMSO alone as untreated control for 8 or 16 h, harvested, washed with cold phosphate buffered saline (PBS) and then directly used for RNA extraction. Two micrograms of total RNA were extracted from HepG2 cells by the Total RNA Mini Kit (R6834-02, Omega Bio-Tek, Inc., Norcross, Ga., USA). RNA was reverse-transcribed into cDNA using the High-Capacity cDNA Reverse Transcription Kit (4368814, Thermo Fischer Scientific, Inc.) according to the manufacturer's protocol. FXR target gene expression was evaluated by quantitative real time PCR analysis with a StepOnePlus™ System (Life Technologies, Carlsbad, Calif., USA) using PowerSYBRGreen (Life Technologies; 12.5 μL per well). The primers are listed in the supporting information. Each sample was set up in duplicates and repeated in at least three independent experiments. The expression was quantified by the comparative ΔΔCt method and glycerinaldehyde 3-phosphate dehydrogenase (GAPDH) served as reference gene. Results (expressed as mean*SEM % change in expression compared to vehicle set as 100%; n≥4): BSEP: DMSO: 100; 1b (50 μM): 557±28; 57 (0.1 μM): 216±18; 57 (1 μM): 222±20. SHP: DMSO: 100; 1b (50 μM): 368±35; 57 (0.1 μM): 242±61; 57 (1 μM): 317±78. CYP7A1: DMSO: 100; 1b (50 μM): 34±12; 57 (0.1 μM): 50±7; 57 (1 μM): 52±8. PPARα: DMSO: 100; 1b (50 μM): 289±59; 57 (0.1 μM): 170±11; 57 (1 μM): 211±10. SREBP1c: DMSO: 100; 1b (50 μM): 45±7; 57 (0.1 μM): 49±17; 57 (1 μM): 36*12. FAS: DMSO: 100; 1b (50 μM): 34±14; 57 (0.1 μM): 22±8; 57 (1 μM): 38*15. FGF19: DMSO: 100; 1b (50 μM): 407±42; 57 (0.1 μM): 309±101; 57 (1 μM): 325±77. PDK4: DMSO: 100; 1b (50 μM): 284±50; 57 (0.1 μM): 255±54; 57 (1 μM): 226±57. FABP1: DMSO: 100; 1b (50 μM): 249*17; 57 (0.1 μM): 183±34; 57 (1 μM): 194±42. CD36: DMSO: 100; pioglitazone (1 μM): 353±43; 57 (1 μM): 119±33; 57 (10 μM): 129±42. FAM3A: DMSO: 100; pioglitazone (1 μM): 310±66; 57 (1 μM): 112±12; 57 (10 μM): 142±7.

Cellular sEH Assay

Quantification of cellular sEH metabolic activity was performed as described by Zha et al.⁵³ Accordingly, 1 μg of total cell homogenate from HepG2 cells (diluted in 100 μl of PBS containing 0.1 mg/ml BSA) was incubated with varying concentrations of 57, N-cyclohexyl-N′-(4-iodophenyl)urea (CIU, 10 μM)³⁶ as positive control or vehicle (DMSO in a final concentration of 1%) as negative control for 15 min at 37° C. 25 ng (+)14(15)-EET-d11 (Cayman Chemical, Ann Arbor, USA) was added and incubation was continued for additional to min at 37° C. A blank test was performed with PBS (containing 0.1 mg/ml BSA) treated likewise. The reactions were stopped by adding 100 μl of ice cold methanol. After centrifugation (2000 rpm, 4° C., 5 min), supernatants were analyzed by LC-MS/MS and the amounts of (±)14(15)-EET-d11 and the corresponding (±)14(15)-DHET-d11 were determined. Quantification of (±)14(15)-EET-d11 and (±)14(15)-DHET-d11 from supernatants was performed by LC-MS according to the procedure reported for quantification of EETs and DHETs from mouse plasma (see below). Results (expressed as mean (±)14(15)-EET-d11/(±)14(15)-DHET-d11 ratio±SEM; n=3): blank (w/o cells): 499±123; DMSO (1%): 63±16; CIU (10 μM): 519±55; 57 0.001 μM: 191±7; 0.01 μM: 396±65; 0.1 μM: 442±7; 1 μM: 602±56; 10 μM: 614±96.

Animal Study

Animals and compound application: 9 male C57BL/6JRj mice (23-26 g body weight, purchased from Janvier Labs, France) were used in the present study. The animals were housed in a temperature-controlled room (20-24° C.) and maintained in a 12 h light/12 h dark cycle. Food and water were available ad libitum. The in life phase was performed by the contract research organization Pharmacelsus (Saarbrücken, Germany). All experimental procedures were approved by and conducted in accordance with the regulations of the local Animal Welfare authorities (Landesamt für Gesundheit und Verbraucherschutz, Abteilung Lebensmittel-und Veterinärwesen, Saarbrücken). Six animals received a single oral dose of 10 mg/kg body weight of dual modulator 57 in water containing 1% HPMC/Tween 80 (99:1). Three animals received the vehicle (water containing 1% HPMC/Tween 80 (99:1)). All animals behaved normal throughout the study and showed no adverse effects.

Blood and liver sampling: At six time points (15 min, 30 min, 60 min, 120 min, 240 min and 480 min after application of 57), blood (20 μl) of the six restrained and conscious mice was obtained from the lateral tail vein. For the last time-point (480 min), mice were anesthetized under isoflurane and blood (˜500 μl) was obtained by retro-orbital puncture. A part of the blood was centrifuged (6000 rpm, 10 min, +4° C.) to obtain plasma for quantification of EET/DHET ratio and stored at −80° C. until further evaluation. For liver collection, mice were sacrificed by cervical dislocation after the last blood sampling (8 h post dose). Complete liver was immediately snap-frozen and stored at −80° C. until further evaluation. Plasma and liver were equally obtained from three control mice which received oral application of the vehicle (1% HPMC/Tween 80 (99:1)).

Quantification of 57 from blood samples: Calibration samples: Stock solutions of the test items (1 mg/ml in DMSO) were diluted in DMSO to a final concentration of 200 μg/ml (start solution). Further working solutions were prepared by dilution of the working solutions in DMSO. Individual stock solutions were used for preparation of calibration standards and QCs. Calibration standards and QCs were prepared by spiking 20 μl of drug free blank blood with 2.4 μl working solution. Accordingly, unknown samples, zero samples and blanks were spiked with 2.4 μl DMSO. The calibration standards and quality controls were prepared in duplicates. A volume of 40 μl acetonitrile containing the internal standard (Griseofulvin, 600 ng/ml) was added to 22.4 μl of unknown sample, zero sample, calibration standard and QC sample. Acetonitrile without internal standard was added to blank samples. All samples were vigorously shaken and centrifuged for to minutes at 6000 g and room temperature. The particle free supernatant (50 μl) was diluted with an equal volume water. An aliquot was transferred to 200 μl sampler vials and subsequently subjected to LC MS with an injection volume of 15 μl. LC-MS analysis: The HPLC pump flow rate was set to 600 μl/min and the compounds were separated on a Kinetex Phenyl-Hexyl, 2.6 μm, 50×2.1 mm (Phenomenex, Aschaffenburg, Germany) analytical column with a pre-column (Kinetex Phenyl-Hexyl, SecurityGuard Ultra, 2.1 mm). Gradient elution with water and 0.1% formic acid as aqueous phase (A) and acetonitrile with 0.1% formic acid as organic phase (B) was used: % B (t (min)), 0(0-0.1)-97(0.4-1.7)-0(1.8-3.0). Full scan mass spectra were acquired in the positive ion mode using syringe pump infusion to identify the protonated quasi-molecular ions [M+H]⁺. Auto-tuning was carried out for maximizing ion abundance followed by the identification of characteristic fragment ions using a generic parameter set: ion-transfer-capillary temperature 350° C., capillary voltage 3.8 kV, collision gas 0.8 mbar argon, sheath gas, ion sweep gas and auxiliary gas pressure were 20, 2 and 8 (arbitrary units), respectively. Pharmacokinetic analysis was performed applying a non-compartment model using the Kinetica 5.0 software (Thermo Scientific, Waltham, USA).

Quantification of FXR target gene mRNA from mouse livers: Hepatocyte isolation of mouse liver tissue for RT-qPCR: To homogenize the liver samples, one third of each liver was placed on one Falcon™ Cell Strainer with 40 μm pore size (BD Bioscience, Erembodegem, Belgium) in a 50 mL Falcon tube. Every tissue was rinsed with PBS buffer containing 10% FCS and 1% PenStrep and pressed through the cell strainer until 5 mL cell suspension had been collected. The samples were centrifuged at 1200 rpm for to min at 4° C. The supernatant was discarded and the pellets were washed with 5 mL cold PBS buffer and again centrifuged at 1200 rpm for 10 min at 4° C. After discarding the supernatant, the cell pellets were re-suspended in 1 mL PBS and total RNA was extracted using the E. Z. A. Total RNA Kit I (Omega bio-tek Inc., Narcoss, Ga., USA) following the Animal Tissue Protocol. The extracted RNA was used for qRT-PCR and equally treated as described for mRNA quantification from HepG2 cells (see above). PCR primers for the murine genes are listed in the supporting information. Results (expressed as mean±SEM % change in expression compared to vehicle set as 100%; n=6 for 57, n=3 for vehicle): BSEP: vehicle: 105±24; 57 (10 mg/kg): 312±103. SHP: vehicle: 108+29; 57 (10 mg/kg): 410±147. CYP7A1: vehicle: 106±25; 57 (10 mg/kg): 70±11. SREBP1c: vehicle: 82±14; 57 (10 mg/kg): 17±10. FGF15: vehicle: 119±15; 57 (10 mg/kg): 254±9. FATP: vehicle: 101±1; 57 (10 mg/g): 105±5.

EET/DHET ratio analysis from mouse plasma samples (Determination of Epoxyeicosatrienoicacids (EETs) and their metabolites Dihydroxyepoxyeicosatrienoicacids (DHETs) by LCMS/MS): 8.9-EET, 11.12-EET and their dehydro-metabolites content of the extracted samples were analyzed employing liquid chromatography tandem mass spectroscopy (LC-MS/MS). The LC-MS/MS system comprised an API 5500 QTrap (AB Sciex, Darmstadt, Germany), equipped with a Turbo-V-source operating in negative ESI mode, an Agilent 1200 binary HPLC pump and degasser (Agilent, Waldbronn, Germany) and an HTC Pal autosampler (Chromtech, Idstein, Germany) fitted with a 25 μL LEAP syringe (Axel Semrau GmbH, Sprockhövel, Germany). High purity nitrogen for the mass spectrometer was produced by a NGM 22-LC/MS nitrogen generator (cmc Instruments, Eschborn, Germany). All compounds were obtained from Cayman Chemical (Ann Arbor, Mich., USA). Stock solutions with 2500 ng/ml of all analytes were prepared in methanol. Working standards with a concentration range of 0.1-250 ng/ml were obtained by further dilution for epoxyeicosatrienoic acids and their dehydro-metabolites. Extraction of mouse plasma samples was performed by liquid-liquid-extraction. 150 μl of sample/matrix homogenate were gently mixed with 20 μl of internal standard (8.9-EET-d8 and 11.12-EET-d8 with a concentration of 200 ng/ml in methanol), and were extracted twice with 600 μL of ethyl acetate. To prepare samples for standard curve and quality control, 150 μL PBS were added further 20 μL methanol 20 μL working standard and 20 μL internal standard were added instead of 150 μl of matrix homogenates. The organic solvent was evaporated at a temperature of 45° C. under a gentle stream of nitrogen. The residues were reconstituted with 50 μL of methanol/water (50:50, v/v), centrifuged for 2 minutes at 10000 g and then transferred to glass vials (Macherey-Nagel, Düren, Germany) prior to injection into the LC-MS/MS system. For the chromatographic separation a Gemini NX C18 column and precolumn were used (150 mm×2 mm i.d., 5 μm particle size and 110 Å pore size from Phenomenex, Aschaffenburg, Germany). A linear gradient was employed at a flow rate of 0.5 ml/min mobile phase with a total run time of 17.5 minutes. Mobile phase was A water/ammonia (100:0.05, v/v) and B acetonitrile/ammonia (100:0.05, v/v). The gradient started from 85% A to 10% within 12 min this was held for 1 min at 10% A. Within 0.5 min the mobile phase shifted back to 85% A and was held for 3.5 min to equilibrate the column for the next sample. The injection volume of samples was 20 μL. Quantification was performed with Analyst Software V 1.5.1 (Applied Biosystems, Darmstadt, Germany) employing the internal standard method (isotope-dilution mass spectrometry). Ratios of analyte peak area and internal standard area (y-axis) were plotted against concentration (x-axis) and calibration curves were calculated by least square regression with 1/c² weighting. Results (expressed as mean ratio±SEM; n=6 for 57, n=3 for vehicle): 8,9-EET/8,9-DHET: vehicle: 0.40±0.03; 57 (10 mg/kg): 0.63±0.03. 11,12-EET/11,12-DHET: vehicle: 0.15±0.01; 57 (10 mg/kg): 0.25±0.03.

1. A compound having the formula I: 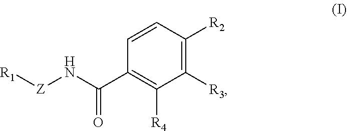
wherein R₁, R₂, R₃ and R₄ are independently selected from H, an unsubstituted, monosubstituted, or polysubstituted C₁-C₁₈ alkyl or heteroalkyl, wherein said alkyl is straight, branched or cyclic, a unsubstituted, monosubstituted or polysubstituted C₁-C₁₈ alkenyl or heteroalkenyl, wherein said alkenyl is straight, branched or cyclic, an unsubstituted, monosubstituted, or polysubstituted aryl or heteroaryl, an unsubstituted, monosubstituted, or polysubstituted benzyl group, an acyl group, such as formyl, acetyl, trichloroacetyl, fumaryl, maleyl, succinyl, benzoyl, or acyl groups being branched, heteroatom-substituted or aryl-substituted, a sugar or another acetal, and a sulfonyl group, and/or R₂, R₃ and/or R₄ form together a nonsubstituted, monosubstituted, or polysubstituted ring, preferably an aromatic ring;
Z is C with or without any substitution;
or an isomer, prodrug, or derivative thereof, or a pharmaceutically acceptable salt or solvate of these compounds.

2. The compound according to claim 1, wherein R₂ is C₁-C₁₀ alkyl, preferably a branched alky, more preferably —C(CH₃)₃, preferably R₃ is H, —OH or —OMe, and preferably R₄ is H, —OH or —OMe.

3. The compound according to claim 1, wherein R₁ is a mono or polysubstituted aryl.

4. The compound according to claim 3, wherein R₁ is selected from any of the following groups:

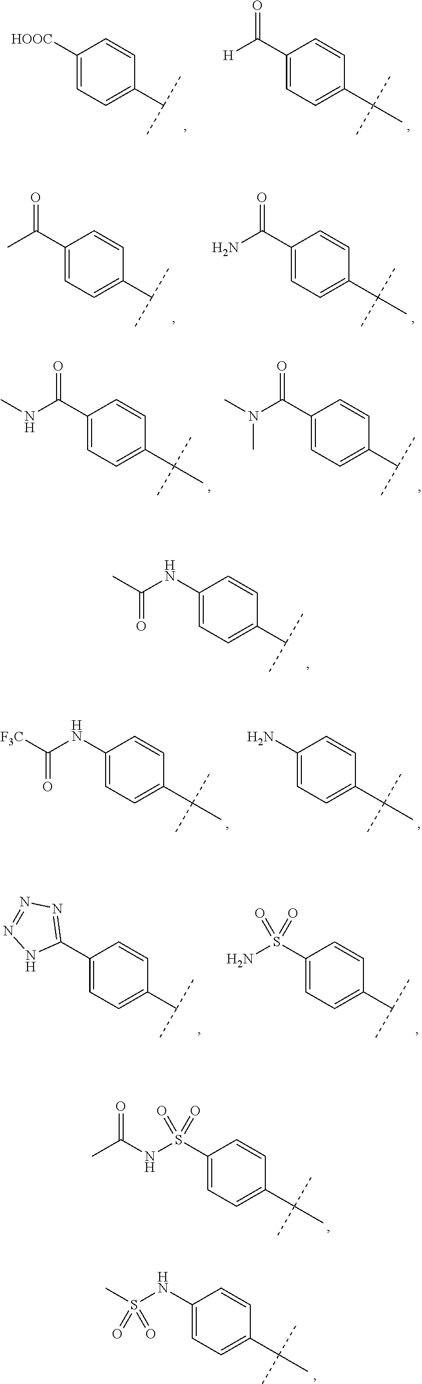

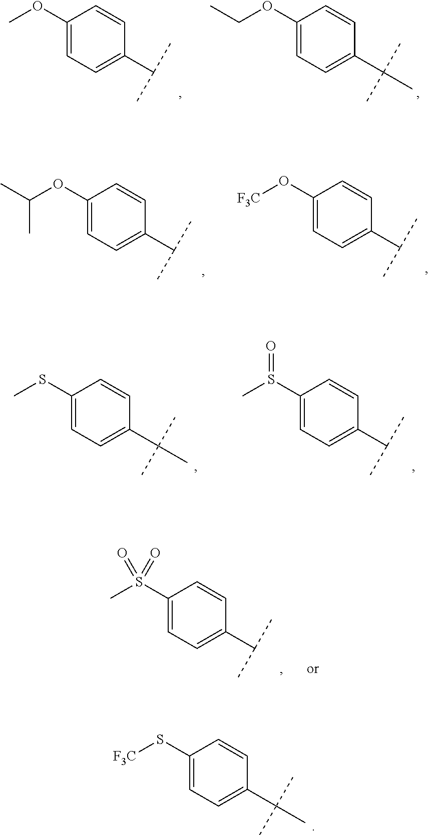

5. The compound according to claim 1, wherein R₁ is selected from the group consisting of: 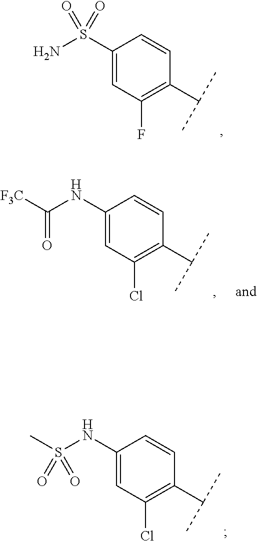
and
wherein Z is C, R₂ is —C(CH₃)₃, and R₃ is H.

6. The compound according to claim 1, wherein R₁ is selected from the group consisting of: 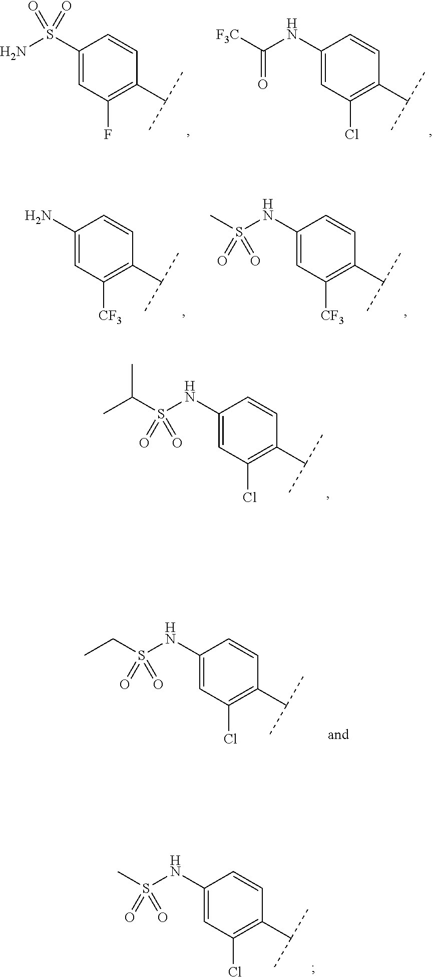
wherein Z is C, R₃ is H or OH, and R₄ is H or OH, in particular R₃ and R₄ are not both OH; and wherein R₂ is selected from —C(CH₃)₃, —N(CH₃)₂, or the R₂ is any of the following structures:

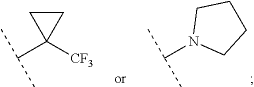

7. The compound according to claim 1, which is a farnesoid X receptor (FXR) agonist and a soluble epoxide hydrolase (sEH) inhibitor.

8. The compound according to claim 1, for use in the treatment of a disease.

9. The compound for use according to claim 8, wherein the disease is a disorder associated with FXR and sEH.

10. The compound for use according to claim 8, wherein the disease is a metabolic disorder, preferably a metabolic disorder caused by or associated with a high-fat diet.

11. The compound for use according to claim 8, wherein the disease is a liver disease, such as non-alcoholic fatty liver disease or non-alcoholic steatohepatitis (NASH).

12. A method for producing a compound according to any claim 1.

13. A pharmaceutical composition comprising a compound according to claim 1, together with a pharmaceutical acceptable carrier and/or excipient.

14. A method for concomitant modulation of FXR and sEH, the method comprising the step of administering to a subject a compound according to claim 1.

15. The method according to claim 14, wherein the subject is suffering from a disease, preferably a metabolic disease.

16. The method according to claim 15, wherein the method is a method of treating the disease of the subject by administering said compound to the subject.

17. The method according to claim 14, wherein modulation is an activation of FXR and inhibition of sEH.

18. The method according to claim 14, wherein administering comprising administering the compound in a therapeutically effective amount to the subject.

19. A method of treating a disease in subject, the method comprising a step of administering to the subject a therapeutically effective amount of the compound according to claim 1.

20. The method according to claim 14, wherein the subject is a mammal, preferably a mouse, rat, donkey, horse, cat, dog, guinea pig, monkey, ape, or preferably is a human patient.

21. The method according to claim 16, wherein the disease is a metabolic disorder, preferably a metabolic disorder caused by or associated with a high-fat diet.

22. The method according to claim 16, wherein the disease is a liver disease, such as non-alcoholic fatty liver disease or non-alcoholic steatohepatitis (NASH).
